(19) United States
(12) Patent Application Publication
(10) Pub. No.: US 2022/0364156 A1
(43) Pub. Date: Nov. 17, 2022

(54) ESTIMATING A QUANTITY OF MOLECULES IN A SAMPLE

(71) Applicant: Biota Technology, Inc., Houston, TX (US)

(72) Inventors: Thomas Ishoey, Encinitas, CA (US); Brian Clement, Irvine, CA (US); Jonathan Sanders, Ithaca, NY (US); Heather Callahan, Escondido, CA (US)

(21) Appl. No.: 17/623,396

(22) PCT Filed: Jun. 24, 2020
(86) PCT No.: PCT/US2020/039279

§ 371 Date: Dec. 28, 2021

Related U.S. Application Data
(60) Provisional application No. 62/868,460, filed on Jun. 28, 2019.

(51) Int. Cl. C12Q 1/689

(52) U.S. Cl. CPC C12Q 1/689; C12Q 1/6869

(57) ABSTRACT

A synthetic molecule can be added to a sample at a specified concentration to accurately and/or precisely quantify target molecules included in the sample. The synthetic molecule can include a number of nucleotides. Some of the regions of the synthetic molecule can include sequences that correspond to primers used in an amplification process and other regions of the synthetic molecule can include sequences that are machine-generated. In implementations, an initial number of target molecules included in the sample can be determined based on a number of the target molecules included in an amplification product in relation to the number of synthetic molecules added to the sample.

### PRIORITY

This application claims priority to U.S. Provisional Patent Application No. 62/868,460, filed Jun. 28, 2019, which is incorporated by reference herein in its entirety.

### TECHNICAL FIELD

The present application relates generally to the technical field of DNA sequencing, and, in one specific example, to methods, systems, and computer-readable mediums having instructions thereon for using information derived from a presence of a synthetic molecule in a sample to determine a quantity of a target molecule in the sample.

### BACKGROUND

Reliably quantifying the abundance of bacteria in samples with very low input biomass, or in DNA extracts with very small quantities of DNA, is a challenging problem. Characterization of very low-abundance bacterial communities is of growing interest for many sample types of commercial or medical interest. These include surfaces analyzed for forensic trace evidence; verification of sterilization of interplanetary spacecraft; human tissues that are typically aseptic or nearly so, such as blood, brain, and uterus; and low-biomass environments of commercial relevance, such as the deep-subsurface environments associated with petroleum reservoirs. The problem of quantification is especially relevant to the field of microbial population analysis via DNA sequencing, as trace microbial contaminants in the reagents used for sequencing can be present at quantities approaching those of the target population, confounding analysis.

### BRIEF DESCRIPTION OF THE DRAWINGS

Some implementations are illustrated by way of example and not limitation in the figures of the accompanying drawings.

FIG. 1 is an example framework to quantify molecules in a sample according to some implementations.

FIG. 2 is an example framework to quantify molecules in samples obtained from multiple sources according to some implementations.

FIG. 3 is a block diagram of an example system to quantify molecules included in a sample according to some implementations.

FIG. 4 is a flow diagram illustrating an example process to quantify molecules included in a sample according to some implementations.

FIG. 5 is a flow diagram illustrating another example of a process to quantify molecules included in a sample according to some implementations.

FIG. 6 is a flow diagram illustrating an example process to quantify molecules included in a sample with specified limits of detection according to some implementations.

FIG. 7 is a flow diagram illustrating an example process to quantify molecules included in samples obtained from multiple sources according to some implementations.

FIG. 8 is a visual representation of the process of adding synthetic spike-in molecules to raw samples to generate more accurate assessments of their quantities in the samples relative to conventional techniques.

FIGS. 9A and 9B are charts showing an example of how quantity estimates of a target molecule may correspond to expected input (ZymoBIOMICS Microbial Community Standard) values across three orders of magnitude, with homoskedastic variance of estimates in log-transformed space.

FIG. 10 is a chart showing an example of how estimates of an absolute starting copy number in low-abundance natural subsurface communities may show linear response in estimates down to very high dilutions. Samples with higher variation identify outliers for further investigation.

FIG. 11 is a chart showing how the outlier abundance measurement in FIG. 10 can be interpreted via the specific sequence types observed in the outlier sample and compared to available databases highlighting the utility over qPCR and other methods where the sequence information is not known.

FIG. 12 is a chart showing how Spearman rank correlation may reveal a set of putative contaminant sequences, which (also consistent with being contaminants) are present across all sample types and are especially abundant in no-template control “blank” samples.

FIG. 13 is a chart showing an example principal coordinate analysis ordination of sample similarity measures.

FIG. 14 is a chart showing how, after removing reagent contaminants detected with the disclosed spike-in sequences, a trend of low biomass samples tending to be more similar to one another as the shared reagent contaminant sequences made up increasingly larger proportions of the samples may be largely eliminated.

### DETAILED DESCRIPTION

In the following description, for purposes of explanation, numerous specific details are set forth in order to provide an understanding of various implementations of the present subject matter. It will be evident, however, to those skilled in the art that various implementations may be practiced without these specific details.

Techniques for quantifying bacteria in low concentrations broadly fall into two categories: measurements of cells (e.g., using microfluidics or flow cytometry) and molecular measurement of nucleic acids (e.g., via a quantitative polymerase chain reaction).

Cell measurement techniques are typically lower-throughput than molecular techniques, and they are relatively capital-intensive. For example, quantitative flow cytometry has been used to measure free-floating (planktonic) bacterial cells in low concentrations (e.g., with a detection limit of around 200 cells/mL). Recently developed experimental microfluidics platforms have also demonstrated some sensitivity (e.g., down to ˜20 cells/mL). However, this sensitivity requires highly specific cell-surface recognition molecules, such as targeted antibodies, which are of general use for detection of targeted organisms but of more limited use for broad estimates of abundance. Furthermore, both microfluidics and flow cytometry require intact cells in a suspension, limiting their application to biofilms or host-tissue-associated samples, and they require special sample storage and preparation.

Because nucleic acid amplification like Polymerase Chain Reaction (PCR) or Multiple Displacement Amplification (MDA) amplifies very low quantities of DNA using exponential replication, it has the potential to detect molecules at very low concentrations. It is also the underlying technology used for most microbial community sequencing. Use of highly conserved regions of the universal Small Subunit Ribosomal RNA gene (SSU-rRNA) as polymerase priming sites allows PCR approaches to target relatively broad taxonomic ranges of bacteria and archaea. Quantitative PCR (qPCR) instruments, while fairly expensive, are widely adopted in molecular biology laboratories. And because it is an application of the same enzymatic molecular technique used to prepare samples for DNA sequencing, using qPCR for quantification entails little sample preparation overhead compared to flow-cytometric or microfluidics-based approaches. For these reasons, qPCR is the most broadly applied method for absolute quantification of microbes in most sample types.

Conventional qPCR methods use a serially-diluted standard curve of known target molecule concentrations to interpolate unknown sample concentrations based on the time to amplification past some fluorescently measured critical threshold. Conventional qPCR can be sensitive when used with carefully designed amplification primers in combination with specific probes, with sensitivities approaching single target molecules per μL. However, quantification of unknown bacterial communities requires the use of degenerate primers and non-probe-based fluorescence measurement, which in practice limits the sensitivity of the assay (e.g., typical lower limits of detection (LoD) are frequently around 50-100 copies/reaction). Different bacterial SSU-rRNA genes also have different amplification efficiencies, which influence the inferred sample concentration depending on the relative efficiency of amplification of the standard curve. Cross-reactivity of universal bacterial primers with eukaryotic SSU-rRNA genes also raises this LoD in host-associated samples.

Newer qPCR techniques use recently commercialized microfluidics devices to perform PCR reactions in individual micro-droplets, an approach typically referred to as digital droplet PCR, or ddPCR. Because this approach uses endpoint amplification in conjunction with the Poisson distribution of target molecules across droplets, rather than amplification kinetics, it does not require a standard curve, and so is less sensitive to variations in amplification efficiency or limited cross-reactivity with off-target genes. The dynamic range of ddPCR is dictated by the number of droplets analyzed, and thus typically narrower than conventional PCR (e.g., ˜5 vs ˜8 log units), but with a substantially improved LoD in broad-based bacterial assays, on the order of 10-15 copies/reaction. However, ddPCR requires expensive dedicated equipment and is substantially more expensive and lower-throughput than conventional qPCR, and both methods require an entirely separate protocol to be run in parallel to sequencing.

Spiked-in synthetic DNA may be used as internal calibration standards for estimation of sequencing error profiles, detection of sample cross-contamination, and estimation of abundance. Synthetic DNA has the advantage of being ‘read’ in the same sequencing step during which community profile is estimated, thus, it does not require any additional equipment or separate laboratory steps. In conventional techniques, synthetic DNA has been obtained that includes sequences from biological organisms not expected to be present in the target samples, as well as computer-generated sequences. But, this approach has the disadvantage of limiting the range of sample types that can be used with a given synthetic molecule, and prior evidence for this approach has not demonstrated sensitivity for quantification of diluted samples. Synthetic DNA molecules matching PCR primer binding sites can be used with many natural samples and have been demonstrated to be effective for quantification (e.g., with limits of detection between 87 and 246 copies/reaction) without adversely affecting the estimates of community diversity. However, these previous methods used an internal standard curve of different synthetic molecules at different abundances, requiring synthesis and preparation of a more complex spike-in mixture and the dedication of comparatively large proportion of total sequencing effort to ensure species from the target sample fell within the abundance range of the spike-in curve.

Thus, using conventional techniques, it is possible to generate some data about a target molecule in a sample that contains a low quantity of the target molecule. However, gene sequencing by itself does not provide information about the quantity of the target molecule in the sample, and existing techniques for measuring the quantity of the target molecule in the sample may fall short of a desired accuracy (e.g., because the conventional techniques are sensitive to confounding by contaminant particles in the sample), such as providing an absolute quantity of the target molecule.

In example implementations, a synthetic molecule can be added to a sample at a specified concentration to accurately and/or precisely quantify target molecules included in the sample. The synthetic molecule can include a number of nucleotides and the arrangement of nucleotides included in the synthetic molecule can be represented as a sequence of nucleotides. In various implementations, the synthetic molecule added to the sample can be referred to herein as a “spike-in” or a “spike-in molecule”. In implementations, the quantification of target molecules can be performed with respect to samples that have a low biomass. Additionally, contaminants included in the sample can also be identified using the synthetic molecule. In this way, the lower limit of detection is improved in comparison to conventional techniques, including qPCR, and is much less expensive and labor intensive than conventional techniques, including ddPCR.

As used herein, biomass can refer to the number of DNA-containing biological cells in a sample. The number of DNA-containing biological cells can be estimated via DNA concentration or sequence copy number. Additionally, as used herein, low biomass can refer to sample types typically containing orders of magnitude fewer cells than are found in rich microbial habitats. Rich microbial habitats can include human gut, skin, and saliva having on the order of 10¹⁰ cells/mL and soil having on the order of 10¹⁰ cells/g. Low biomass habitats can include oligotrophic seawater having on the order of 10⁵ cells/mL and below.

The synthetic molecule can include regions that correspond to primers used for targeted gene amplification and sequencing that are interspersed with regions of machine-generated nucleotide sequences that are not found in nature. A known quantity of this synthetic molecule is added (“spiked in”) to the reagents used for amplification of the target molecules. Because these synthetic molecules include regions including the computer-generated nucleotide sequences that are not found in natural target molecules, they can be identified in the sequence output, and information from their abundance relative to the abundances of natural sequences derived from the sample is used to 1) estimate the initial concentration of target molecules in the sample, and 2) identify potential contaminant molecules present in the reagents.

This disclosure provides for a precise quantification of diverse genes at relatively low target concentrations (on the order of less than 50 molecules per μL down to 1 molecule per μL) and provides for an absolute quantification simultaneously with amplification and sequencing operations. The techniques described in implementations herein can be performed at relatively low per-unit additional cost relative to conventional techniques, and with minimal additional labor, when performed in conjunction with existing sequencing projects. Further, the implementations described herein can be performed with no additional equipment (i.e. quantitative PCR machines) and no additional enzymatic processes (i.e. additional PCR reactions) when added to an existing workflow of sequencing projects. The use of the synthetic molecules described in implementations herein can also be used as an internal reference to remove contaminating molecules or nucleotide sequences arising from reagents used in the amplification process.

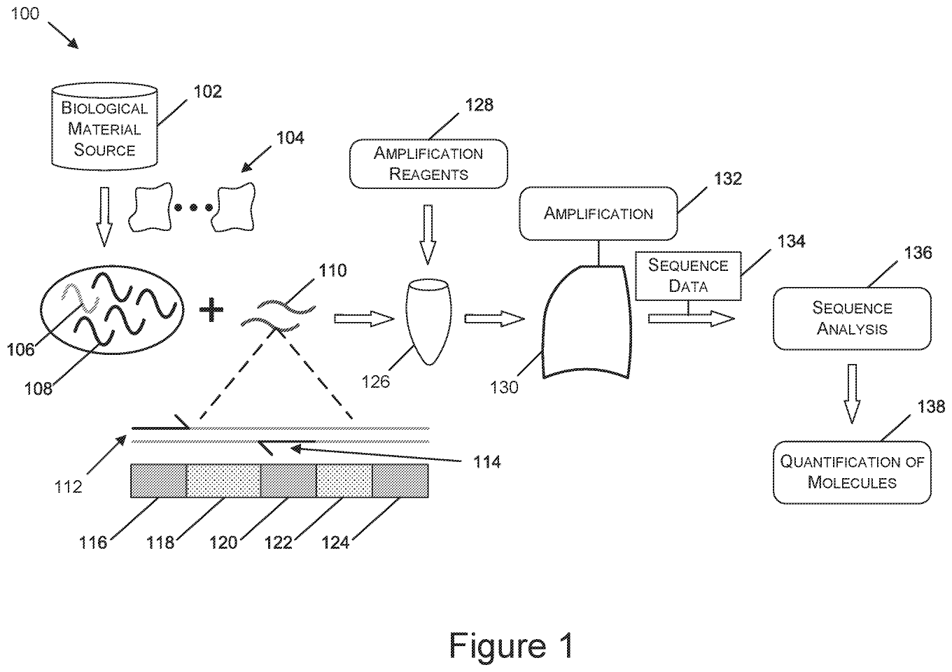

FIG. 1 is an example framework 100 to quantify molecules in a sample according to some implementations. The framework 100 includes a biological material source 102 from which amounts of biological material 104 can be obtained. The biological material source 102 can include a number of sources, in some implementations. In various implementations, the biological material source 102 can include a liquid. Additionally, the biological material source 102 can include a solid. In further implementations, the biological material source 102 can include a gas.

The biological material source 102 can include a subterranean environment where oil and natural gas can be located. In these situations, the biological material 104 can include a fossil fuel-based substance or a natural gas substance. The biological material source 102 can also include an agricultural environment. Thus, in these scenarios, the biological material can include portions of crops or soil. Further, the biological material source 102 can include an environment where materials are gathered for forensics purposes. In these instances, the biological material 104 can include substances related to a human body (e.g., hair, saliva, skin, and the like), or materials that include substances related to the human body (e.g., clothing, personal care products, etc.). In still other examples, the biological material source 102 can include an environment where contamination of food can take place or contamination of air can take place. In these implementations, the biological material 104 can include a food product, a substance related to a food product (e.g., utensils, plates, bowls, etc.), or particulates from the air.

Genetic molecules can be extracted from the biological material 104. For example, deoxyribonucleic acid (DNA) or ribonucleic acid (RNA) can be extracted from the biological material 104. The genetic molecules can correspond to different biological organisms included in the biological material 104. In the illustrative example of FIG. 1, a first genetic molecule 106 and a second genetic molecule 108 can be extracted from the biological material 104.

In various implementations where quantification of the first genetic molecule 106 and quantification of the second genetic molecule 108 are to take place, samples can be produced that include an amount of the first genetic molecule 106 and the second genetic molecule 108 taken from the biological material 104. In example implementations, the samples can be used in the quantification of the first genetic molecule 106 and the second genetic molecule 108. The samples can be prepared by producing a mixture that includes an amount of the first genetic material 106, an amount of the second genetic material 108, and an amount of a synthetic molecule 110. The synthetic molecule 110 can include a number of regions of nucleotide sequences with at least a portion of the regions being complementary to primers used in the amplification of genetic molecules. In the illustrative example of FIG. 1, the synthetic molecule 110 can include regions that correspond to a first primer 112 and a second primer 114.

The synthetic molecule 110 can also include a number of other regions of nucleotide sequences that are computer-generated sequences of nucleotides. The computer-generated sequences of nucleotides can be produced using random number generator techniques or pseudo-random number generator techniques. The computer-generated sequences of nucleotides can be produced such that fewer than a threshold number of adjacent nucleotides in the computer-generated sequences have identity with portions of nucleotide sequences of biological organisms. That is, the computer-generated sequences can have less than a threshold amount of identity with respect to nucleotide sequences of biological organisms over a specified number of nucleotides, such that the computer-generated sequences can be readily distinguished from any known naturally-occurring nucleotide sequence.

In illustrative examples, the synthetic molecule 110 can include a first region 116 that corresponds to the first primer 112 and a second region 118 that is a first computer-generated region. Additionally, the synthetic molecule 110 can include a third region 120 that corresponds to the second primer 114 and a fourth region 122 that is a second computer-generated region. Further, the synthetic molecule 110 can include a fifth region 124 that can correspond to another primer used in the amplification of genetic molecules, in some situations, while in other implementations, the fifth region 124 can include a computer-generated region. In various illustrative examples, the first primer 112 can include a 3′ primer, while the second primer 114 can include a 5′ primer. In example implementations, the number of nucleotides included in the regions 116, 118, 120, 122, 124 can vary. In illustrative examples, the first region 116 and the third region 120 can individually have from 25 to 300 nucleotides, from 50 to 250 nucleotides, or from 100 to 200 nucleotides. In some examples, the computer-generated regions 118, 122 can have a greater number of nucleotides than the regions 116, 120. To illustrate, the computer-generated regions 118, 122 can individually have from 50 to 5000 nucleotides, from 100 to 4000 nucleotides, or from 250 to 2500 nucleotides.

To produce the samples used in the amplification of genetic molecules, an amount of the biological material 104 and an amount of the synthetic molecule 110 can be added to a container 126. Additionally, amplification reagents 128 can be added to the container 126. The amplification reagents 128 can include primers used in the amplification process, buffer solutions, one or more enzymes, nucleotides, combinations thereof, and so forth. In various examples, a mixture can be produced in the container 126 and dividing into a number of different portions. The portions can be samples that are provided to an amplification machine 130 that performs an amplification process 132 with respect to the genetic molecules included in the samples and with respect to the synthetic molecule 110. In illustrative examples, the amplification process 132 can include a polymerase chain reaction (PCR) process. The PCR process performed by the amplification process 132 can be a traditional PCR process that does not include a real-time PCR process or a quantitative PCR process. In additional illustrative examples, the amplification process 132 can include an isothermal amplification process, such as a multiple displacement amplification (MDA) process.

The amplification process 132 can produce an amplification product that includes an amplified quantity of the first genetic molecule 106, an amplified quantity of the second genetic molecule 108, and an amplified quantity of the synthetic molecule 110. In implementations, the amplified quantity of a molecule included in the amplification product can be thousands up to millions more of a molecule than the original quantity of molecules included in the sample being amplified.

The amplification product produced by the amplification machine 130 can be sequenced to produce sequence data 134 that indicates nucleotide sequences that correspond to the various molecules included in the amplification product. In some cases, the amplification machine 130 can perform the sequencing process, while in additional scenarios, a separate machine (not shown) can perform the sequencing process. The sequencing process can produce data that indicates individual nucleotides that are located at positions of a molecule where the individual nucleotides are represented by letters that correspond to the specified nucleotide located at a given position.

The sequences included in the sequence data 134 can undergo one or more sequence analysis processes at operation 136. The sequence analysis at operation 136 can include identifying nucleotide sequences corresponding to various molecules included in the samples that were amplified and counting the number of sequences that correspond to the individual molecules. In various examples, the sequences that correspond to a given molecule can be determined based on an amount of identity between the sequences. For example, sequences having at least a threshold amount of identity can be identified as being associated with a same molecule. In additional implementations, a barcode nucleotide sequence can be associated with a given molecule and the nucleotide sequences that include the barcode sequence for the given molecule can be determined to correspond to the same molecule. The bar code sequence can, in example implementations, be added to, or otherwise included in, primers used in the amplification process 132. In additional implementations, the bar code sequence can be identified as a preexisting portion of the nucleotide sequence of a given molecule. In various implementations, the number of nucleotide sequences that correspond to a molecule can be counted and the number of the nucleotide sequences for individual molecules included in the amplification product can be determined.

At operation 138, the quantification of molecules included in the original samples can take place. The quantification of the molecules included in the original samples can be determined using a function that relates the number of nucleotide sequences for a given molecule included in an amplification product to the initial number of the synthetic molecule 110 included in the sample. In various examples, the function can include a ratio that relates the number of nucleotide sequences for a given molecule in an amplification product to the initial number of synthetic molecules 110 included in the original sample that was not amplified. In example implementations, the following formula is used:

((t−s)/s)*n*v

where t is the total number of sequence reads for a sample, s is the number of sequence reads belonging to the synthetic molecule, n is the known number of copies of the synthetic molecule added to the reaction, and v is the volume of sample added to the reaction. Thus, t-s corresponds to the number of non-synthetic molecule sequences included in the sample. For example, in the case described above, n=1000 copies of spike-in are added to PCR reactions containing v=10 μL unknown DNA sample. If a corresponding sample yielded t=10,000 sequence reads, of which s=1000 matched the spike-in, it could be estimated that the original sample contained target molecules at a concentration of approximately 900 copies per μL.

Thus, since the number of the synthetic molecules included in the original sample is a known quantity or a quantity that can be determined with a relatively high level of accuracy, the initial number of molecules of a target molecule included in the original sample can also be determined at a relatively high level of accuracy. In various examples, the number of a target molecule included in an original sample can be determined with a precision of at least 90% and a lower limit of detection from 1 to 100 target molecules in a sample per μL. In additional examples, the number of a target molecule included in an original sample can be determined with a precision of at least 95% and a lower limit of detection from 1 to 50 target molecules in a sample per μL. In further examples, the number of a target molecule included in an original sample can be determined with a precision of at least 98% and a lower limit of detection from 1 to 10 target molecules in a sample per μL. In still other examples, the number of a target molecule can be determined with a precision of at least 98% and a lower limit of detection from 1 to 100 target molecules in a sample per μL. In still further examples, the number of a target molecule can be determined with a precision of at least 95% and a lower limit of detection from 1 to 50 target molecules in a sample per μL.

The number of nucleotide sequences included in the sequence data 134 and the initial number of the synthetic molecule 110 included in an original sample can be used to identify contaminants in the original sample. In illustrative situations, the contaminants may have originated from the amplification reagents 128. In various implementations, a contaminant included in a sample can be determined based on a proportion of the total number of nucleotide sequences at a consistent ratio relative to the synthetic nucleotide sequences across samples. That is, because contaminant DNA molecules derived from amplification reagents and synthetic DNA molecules are always added in equivalent numbers to samples with varying numbers of naturally-occurring DNA molecules, the ratio of contaminant-derived to synthetic-derived sequences will be consistent across samples even as their combined proportion of the total sequences changes as a function of naturally-occurring DNA concentration. Thus, identifying a number of unknown nucleotide sequences in the sequence data 134 that correlate in proportional abundance to the proportional abundance of known synthetic molecule 110 included in the original sample can identify potential contaminants included in a sample.

In example implementations, the contaminant molecules can be determined as dilutions of samples are produced in relation to the amplification product. The dilution process can be performed with a diluent. In one or more examples, the diluent can include water. In additional examples, the diluent can include a buffer solution. In illustrative examples, the dilution can be a 4 times dilution with respect to the amplification product. That is, an amount of diluent is added to an amount of the amplification product such that the amount of the diluted sample is 4 times less than the original amplification product. Additionally, a series of dilutions can take place. In various examples, each dilution in the series can be a greater dilution than the previous sample in the series. In example illustrative implementations, a first dilution can be a 4 times dilution, a second dilution can be a 16 times dilution, a third dilution can be a 64 times dilution, a fourth dilution can be a 256 times dilution, a fifth dilution can be a 1024 times dilution, and so forth. The quantity of target molecules, and other molecules, can be determined with respect to each dilution. As the amount of dilution increases in the series of dilutions, the number of target molecules and synthetic molecules can decrease with contaminant molecules having a relatively constant abundance. Thus, as the dilutions become greater, the contaminant molecules can be identified more readily because fewer target molecules and synthetic molecules are present in the dilutions and the presence of the contaminant molecules can be more readily detected. That is, the amount of contaminants present in each dilution behaves independently of the dilution since the contaminant molecules can be from reagents (e.g., amplification reagents) and not from the original sample that included the target molecules. Further, as the amount of dilution increases, the presence of contaminants can be detected when the relative number of contaminant molecules increases with respect to the number of target molecules and the number of synthetic molecules. In various implementations, the use of the synthetic molecule 110 can provide quality control measures. That is, the presence of the synthetic molecule 110 can set a baseline for identifying contaminants, since contaminants can be present in amounts that are similar to the amounts of the synthetic molecule 110 in an original sample. Thus, in situations where the amount of one or more molecules is greater than or substantially similar to that of the synthetic molecule 110 in the original sample, the presence of one or more contaminants can be detected.

In implementations, biological organisms corresponding to nucleotide sequences can be determined by comparing individual nucleotide sequences included in the sequence data 134 to a library of nucleotide sequences. The library of nucleotide sequences can include nucleotide sequences that have been previously determined to correspond to a number of biological organisms. Thus, a comparison can take place between at least a portion of the nucleotide sequences included in the sequence data 134 and nucleotide sequences included in the library of nucleotide sequences. In situations where the amount of identity between a nucleotide sequence included in the sequence data 134 and a nucleotide sequence included in the library of nucleotide sequences is at least a threshold amount of identity, a determination can be made that the biological organism corresponding to the nucleotide sequence was included in the original sample.

In example implementations, the biological material source 102 can include a human body sample such as blood, saliva, or hair and is analyzed using the workflow in FIG. 1. The output signal in 138 can quantify the target molecule of interest that may include cancer cells or biological markers indicative of cancer. In these scenarios, the quantification of the low biomass target molecules can be used as a liquid-based biopsy for the detection of cancer. Such liquid biopsies enable non-invasive tests to detect cancer cells (circulating tumor cells, CTCs) or DNA shed from tumors (circulating tumor DNA, ctDNA) in the human body. Further examples of this implementation enable cancer detection and monitoring that can be repeated over time for the purpose of diagnosing and quantifying diseases, and also monitoring the progression of a cancer. Aforementioned ctDNA and CTCs are present at very low levels in complex body systems. While it may be difficult, inefficient, and/or costly for conventional techniques to identify these biomarkers, the implementations of the techniques and systems described herein can detect the presence of various low concentration biomarkers with high levels of sensitivities. The implementations of the systems and techniques described herein can detect different cancers, monitor changes in a cancer, detect tumor heterogeneity, find biomarkers, and detect loss of response to various treatments. Some applications of the implementations and techniques described herein can include detection of type 1 diabetes, detection and monitoring of infections, organ transplantation, and noninvasive pregnancy testing.

The biological material source 102 can also include an abundance of microorganisms of interest and can be analyzed using the workflow in FIG. 1. The output signal of operation 138 can quantify the microbiological content of the biological material source and enable a breadth of useful applications in medicine, forensics, pathogen detection, and agricultural analysis. For example, in medicine, the quantification of the microorganisms that can impact the patient before and after transplantation of organ can take place. By using the techniques and systems described herein, quantification of these pathogens can take place with improved precision and detection limits with respect to conventional techniques, which can enable clinicians to provide treatments that can improve patient efficacy or safety of the organ transplant process and that can provide improved administration of medicines or other clinical recommendations.

In additional medical applications, quantification of microorganisms that may be present in a patient before or after the administration of a therapeutic agent can also take place. Applying implementations of techniques and systems described herein can result in quantification of these microorganisms in a more efficient and precise manner than with conventional techniques. In this way, microorganisms can be identified that are resistant to treatment, commonly known as microbial drug resistant or heteroresistance. Such quantification can enable clinicians to provide more appropriate treatment options than without quantification according to techniques and systems described herein, which can improve patient efficacy or safety with enhanced administration of therapies or other clinical recommendations. Further, with respect to pathogen detection, viral loads can be monitored and detected that are often below limits of quantification of conventional techniques. By applying implementations of quantification techniques and systems described herein to these viral loads, clinicians can determine the viral loads present in target samples with improved precision and efficiency and the impact of various treatments to reduce the viral loads to appropriate or safe levels can be identified.

In agricultural analysis, the detection and monitoring of various microorganisms can take place, including bacteria, archaea, viruses, and fungi which can impact food produced in the agricultural industry. Implementations of the techniques and systems described herein can provide enhanced quantification at high throughput and precision of food-borne pathogens in relation to conventional techniques, thereby allowing for improved monitoring of the food supply from production and distribution to consumption by end-users. The improved quantification of molecules based on the systems and techniques described herein can improve the safety of the foods provided to the consumer by more quickly determining the root cause of various contaminations to the food or food supply and provide unique detection of an unknown pathogen for one or more treatments. Implementations of the techniques and systems described herein can also improve agricultural food stability and/or lead to improved resistance of agricultural products to pests and insects.

In example implementations, the biological material source 102 includes a human body sample such as blood, saliva, or hair and is analyzed using the workflow in FIG. 1. The output signal of operation 138 quantifies the genetic content of the target samples to analyze copy number variants or (CNVs). In the human genome, studies indicate over 10% of the genome is composed of CNVs greater than 1 thousand base pairs; over 30% of the genome contains CNVs larger than 100 base pairs. A number of CNVs are linked to genes that cause or impact therapeutic dosing to a patient. Implementing the techniques and systems described herein can provide improved quantification of CNVs in each sample with respect to precision and/or sensitivity. Thus, small changes to CNVs in the biologically sourced material can be detected. Such detection can improve the identification, diagnosis, or treatment of a disease.

In example implementations, the biological material source 102 includes a human body sample such as blood, saliva, hair or cellular biopsy and is analyzed using the workflow in FIG. 1. The output signal of operation 138 quantifies the genetic content of DNA or RNA sequences to analyze low-abundance targets to detect cellular changes for medical research or clinical applications such as non-invasive tests. Analysis of relatively rare sequences using implementations of techniques and systems described herein can enable detection of DNA sequences such as single nucleotide polymorphisms, allele variants or edited RNA with higher sensitivity or specificity than conventional techniques. Such analysis enables improved biological signals with respect to conventional techniques to detect rare variances that are associated with the onset of cancer, new genetic mutations or duplications that cause disease, viral loads (including HIV), non-invasive tests such as prenatal testing of fetal DNA or patient rejection of organ transplants. Analysis of the genetic content using techniques and systems described herein, can be used to provide higher resolution in gene expression measurements than conventional techniques. These gene expressions measurements can be used to analyze the DNA or RNA levels in biological samples which can help characterize DNA methylation, rare mRNA or miRNA, and genetic signatures from single cell biological analysis. The quantification of the above sample types using implementations of systems and techniques described herein, which historically possess low signal to noise ratios, can improve fundamental biological understanding in human medicine as well as clinical decision-making

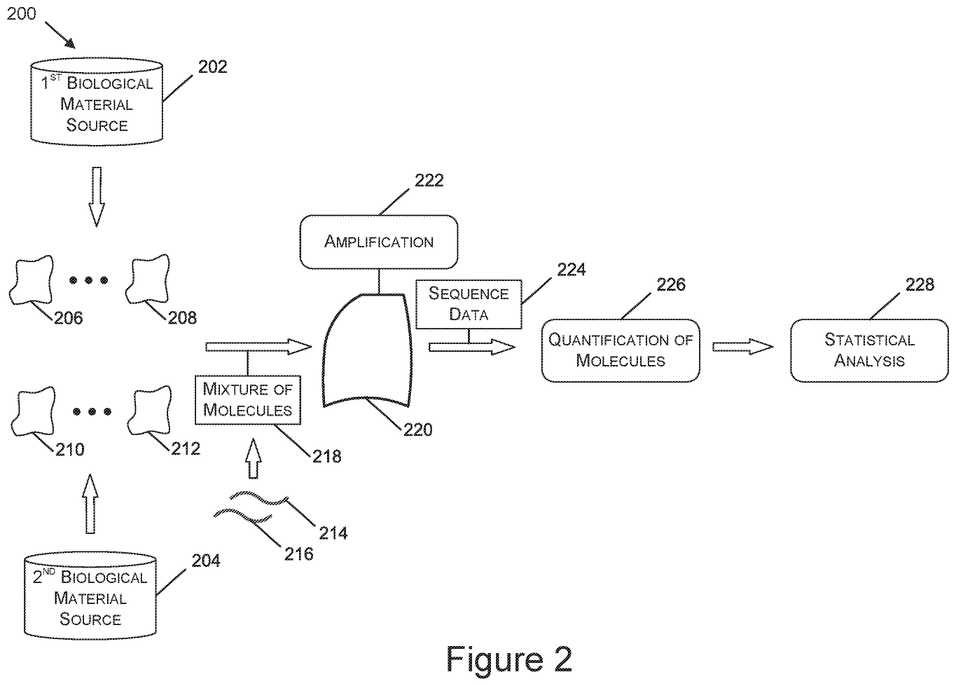

FIG. 2 is an example framework 200 to quantify molecules in samples obtained from multiple sources according to some implementations. The framework 200 includes a first biological material source 202 and a second biological material source 204. A first amount of biological material 206 and a second amount of biological material 208 can be obtained from the first biological material source 202 and a third amount of biological material 210 and a fourth amount of biological material 212 can be obtained from the second biological material source 204.

In implementations, the first biological material source 202 and the second biological material source 204 can be located in the same environment. For example, the first biological material source 202 and the second biological material source 204 can be located in an environment where fossil fuel-based petroleum products and/or natural gas products can be located. In illustrative examples, the first biological material source 202 can be a liquid that includes at least one fossil fuel-based petroleum product and the second biological material source 204 can include rock included in an environment where the fossil fuel-based petroleum product is located. In additional illustrative examples, the first biological material source 202 and the second biological material source 204 can include materials that include human genetic material. To illustrate, the first biological material source 202 can include saliva taken from an individual and the second biological material source 204 can include skin or materials from other body sites of the individual.

Genetic molecules can be extracted from at least one of the amounts of biological material 206, 208, 210, 212. Genetic molecules from one or more of the samples 206, 208, 210, 212 can be mixed with one or more synthetic molecules 214, 216 to produce a mixture of molecules 218. In various implementations, the mixture of molecules 218 can include molecules obtained from the first biological material source 202 and a number of one or more synthetic molecules 214, 216. In additional implementations, the mixture of molecules 218 can include molecules obtained from the second biological material source 204 and a number of one or more synthetic molecules 214, 216. In further implementations, the mixture of molecules 218 can include a number of molecules obtained from the first biological material source 202, a number of molecules obtained from the second biological material source 204, and a number of one or more synthetic molecules 214, 216. In example implementations, the mixture of molecules 218 can include a first number of a first synthetic molecule 214 and a second number of a second synthetic 216 in addition to a number of molecules obtained from at least one of the first biological material source 202 or the second biological material source 204.

One or more samples derived from the mixture of molecules 218 can be placed into a machine 220 and an amplification process can be performed at operation 222 to increase the number of the synthetic molecule 214 and/or 216 and to increase a number of one or more genetic molecules included in the one or more samples. In illustrative examples, the amplification process of operation 222 can include a polymerase chain reaction (PCR) process. The PCR process performed by the amplification process of operation 222 can be a traditional PCR process that does not include a real-time PCR process or a quantitative PCR process. In additional illustrative examples, the amplification process of operation 222 can include an isothermal amplification process, such as a multiple displacement amplification (MDA) process.

A PCR reaction can have three main components: the template, the primers, and enzymes. The template is a single- or double-stranded molecule containing the (sub)sequence of nucleotides to be amplified. The primers are short strands (e.g., less than 40 nucleotides) that define the beginning and end of the region to be amplified. The enzymes include polymerases and thermostable polymerases such as DNA polymerase, RNA polymerase and reverse transcriptase. The enzymes create double-stranded polynucleotides from a single-stranded template by “filling in” complementary nucleotides one by one through addition of nucleoside triphosphates, starting from a primer bound to that template. PCR happens in “cycles,” each of which doubles the number of templates in a solution. The process can be repeated until the desired number of copies is created.

The amplification process of operation 222 can produce an amplification product that includes an amplified quantity of genetic molecules included in the one or more samples and an amplified quantity of the synthetic molecule(s) 214 and/or 216 included in the one or more samples. In implementations, the amplified quantity of a molecule included in the amplification product can be thousands up to millions more of a molecule than the original quantity of molecules included in the sample being amplified.

The amplification product produced by the amplification machine 220 can be sequenced to produce sequence data 224 that indicates nucleotide sequences that correspond to the various molecules included in the amplification product. In some cases, the amplification machine 220 can perform the sequencing process, while in additional scenarios, a separate machine (not shown) can perform the sequencing process. The sequencing process can produce data that indicates individual nucleotides that are located at positions of a molecule where the individual nucleotides are represented by letters that correspond to the specified nucleotide located at a given position.

At operation 226, the quantification of molecules included in the original samples can take place. The quantification of the molecules included in the original samples can be determined using a function that relates the number of nucleotide sequences for a given molecule included in an amplification product to the initial number of the synthetic molecule included in the sample. In various examples, the function can include a ratio that relates the number of nucleotide sequences for a given molecule in an amplification product to the initial number of synthetic molecules included in the original sample that was not amplified.

After quantification of the molecules included in samples obtained from the first biological material source 202 and molecules included in samples obtained from the second biological material source 204, one or more statistical analyses can take place at operation 228. In example implementations, the number of a given molecule included in samples taken from at least one of the first biological material source 202 or the second biological material source 204 can be analyzed over a period of time. Additionally, differences between amounts of target molecules included in samples taken from the first biological material source 202 and amounts of target molecules included in samples taken from the second biological material source 204 can be determined. In illustrative examples, the data obtained indicating changes and/or differences in amounts of target molecules included in samples from the first biological material source 202 and the second biological material source 204 can be analyzed to determine reasons for the changes and/or differences. In some examples, the amount of biomass from which the biological material, such as biological material 206, 208, 210, 210, are obtained can impact the number of a target molecule included in a sample. Other factors can also be determined to cause the changes and/or differences between the number of target molecules included in samples obtained from the first biological material source 202 and the second biological material source 204. To illustrate, environmental conditions, such as temperature, humidity, and pressure differences can cause differences and/or changes in quantities of target molecules included in the samples obtained from the first biological material source 202 and the second biological material source 204. Further, contaminants or other factors can cause the changes and/or differences in quantities of the target molecules included in samples obtained from the first biological material source 202 and the second biological material source 204.

In illustrative implementations, degradation of molecules can be detected using the framework 200. For example, at an initial time (e.g., t0), a mixture of molecules 218 can be produced that includes an amount of a first synthetic molecule 212 and at least one of a number of genetic molecules obtained from the first biological material source 202 or a number of genetic molecules obtained from the second biological material source 204. After a period of time has elapsed, an amount of the second synthetic molecule 216 can be added to the mixture of molecules 218 (e.g., at time t1) and the mixture of molecules 218 can be amplified, at operation 222, and then the amplification product can be sequenced to produce the sequence data 224. In example implementations, the number of the first synthetic molecule 214 added to the mixture of molecules 218 can be at least substantially similar to the number of the second synthetic molecule 216 added to the mixture of molecules 218. In various illustrative examples, the number of the first synthetic molecule 214 added to the mixture of molecules 218 can be the same as the number of the second synthetic molecule 216 added to the mixture of molecules 218.

The quantification of molecules, at operation 226, in these scenarios can determine a number of the first synthetic molecule 214 and a number of the second synthetic molecule 216 included in the mixture of molecules 218 before amplification, at operation 222. In situations where the number of the first synthetic molecule 214 and the number of the second synthetic molecule 216 are within a threshold amount, the statistical analysis, at operation 228, can indicate that little or no degradation of molecules took place within the mixture of molecules 218 from the initial time when the amount of the first synthetic molecule 214 was added to the mixture of molecules 218 to the additional, subsequent time when the amount of the second synthetic molecule 216 was added to the mixture of molecules 218.

In additional scenarios, a quantity of the first synthetic molecule 214 included in the mixture of molecules 218 at the time of amplification of the mixture of molecules 218 can be less than a quantity of the second synthetic molecule 216 included in the mixture of molecules 218 by more than a threshold amount. In these situations, the statistical analysis of operation 228 can indicate that degradation of molecules included in the mixture of molecules 218 has taken place between the initial time when the amount of the first synthetic molecule was added to the mixture of molecules 218 to the additional, subsequent time when the amount of the second synthetic molecule 216 was added to the mixture of molecules 218.

In additional illustrative implementations, a proportion of an unknown mixture of first molecules obtained from the first biological material source 202 and second molecules obtained from the second biological material source 204 can be determined. Both a first number of the first molecules obtained from the first biological material source 202 and a second number of the second molecules obtained from the second biological material source 204 can be quantified using at least one of the first synthetic molecule 214 or the second synthetic molecule 216 in accordance with the framework 200. In example implementations, a first sample including the first molecules obtained from the first biological material source 202 can be used to determine, at operation 226, the first number of the first molecules included in the first sample separately from the determination, also at operation 226, of a second number of the second molecules included in a second sample that includes the second molecules obtained from the second biological material source 204. Subsequently, after mixing samples that include both molecules from the first biological material source 202 and the second biological material source 204, the proportion of the number of first molecules included in the first sample with respect to the number of second molecules included in the second sample can be used to determine the concentration of the molecules obtained from the first biological material source 202 and the molecules obtained from the second biological material source 204 included in the mixture. That is, differences in the quantity of molecules obtained from the first biological material source 202 and the quantity of molecules obtained from the second biological material source 204 that were determined during separate quantification operations can be taken into account when determining the concentration of the molecules in a mixture of samples obtained from the first biological source 202 and the second biological source 204. In this way, the volume associated with each sample used to produce the mixture can also be accounted for when determining the concentrations of molecules included in a mixture of molecules obtained from both the first biological material source 202 and the second biological material source 204.

In further implementations, the presence of one or more target molecules included in a sample can indicate that one or more chemical reactions have taken place. For example, detecting the presence of a genetic molecule in a sample, such as a specified bacteria, can indicate that a biochemical reaction has taken place in the environment (e.g., the first biological material source 202 or the second biological material source 204) from which the sample was obtained. In illustrative implementations, biochemical reactions such as a nitrate reducing reaction, a sulfate reducing reaction, methanogenesis, a hydrocarbon conversion reaction, or a biosurfactant generating reaction, or combinations thereof, can be detected.

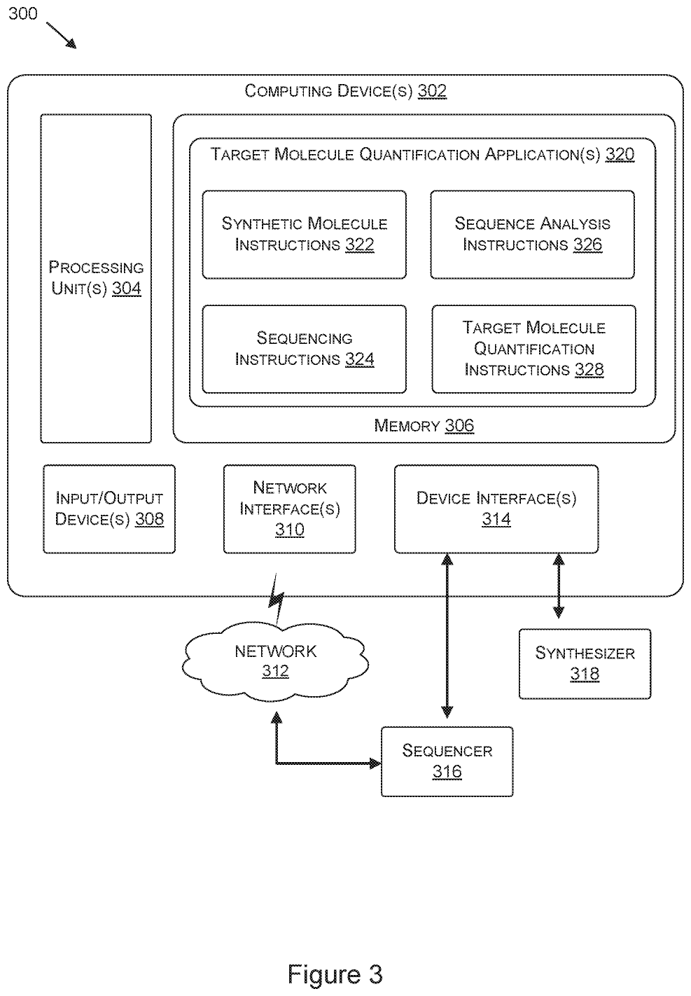

FIG. 3 is a block diagram of an example system 300 to quantify molecules included in a sample according to some implementations. The system 300 can include a computing device 302 that can be implemented with one or more processing unit(s) 304 and memory 306, both of which can be distributed across one or more physical or logical locations. For example, in some implementations, the operations described as being performed by the computing device 302 can be performed by multiple computing devices. In some cases, the operations described as being performed by the computing device 302 can be performed in a cloud computing architecture.

The processing unit(s) 304 can include any combination of central processing units (CPUs), graphical processing units (GPUs), single core processors, multi-core processors, application-specific integrated circuits (ASICs), programmable circuits such as Field Programmable Gate Arrays (FPGA), and the like. In one implementation, one or more of the processing units(s) 304 can use Single Instruction Multiple Data (SIMD) parallel architecture. For example, the processing unit(s) 304 can include one or more GPUs that implement SIMD. One or more of the processing unit(s) 304 can be implemented as hardware devices. In some implementations, one or more of the processing unit(s) 304 can be implemented in software and/or firmware in addition to hardware implementations. Software or firmware implementations of the processing unit(s) 304 can include computer- or machine-executable instructions written in any suitable programming language to perform the various functions described. Software implementations of the processing unit(s) 304 may be stored in whole or part in the memory 306.

Alternatively, or additionally, the functionality of computing device 302 can be performed, at least in part, by one or more hardware logic components. For example, and without limitation, illustrative types of hardware logic components that can be used include Field-programmable Gate Arrays (FPGAs), Application-specific Integrated Circuits (ASICs), Application-specific Standard Products (ASSPs), System-on-a-chip systems (SOCs), Complex Programmable Logic Devices (CPLDs), etc.

The memory 306 of the computing device 302 can include removable storage, non-removable storage, local storage, and/or remote storage to provide storage of computer-readable instructions, data structures, program modules, and other data. The memory 306 can be implemented as computer-readable media. Computer-readable media includes at least two types of media: computer-readable storage media and communications media. Computer-readable storage media includes volatile and non-volatile, removable and non-removable media implemented in any method or technology for storage of information such as computer-readable instructions, data structures, program modules, or other data. Computer-readable storage media includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other non-transmission medium that can be used to store information for access by a computing device.

In contrast, communications media can embody computer-readable instructions, data structures, program modules, or other data in a modulated data signal, such as a carrier wave, or other transmission mechanism. As defined herein, computer-readable storage media and communications media are mutually exclusive.

The computing device 302 can include and/or be coupled with one or more input/output devices 308 such as a keyboard, a pointing device, a touchscreen, a microphone, a camera, a display, a speaker, a printer, and the like. Input/output devices 308 that are physically remote from the processing unit(s) 304 and the memory 306 can also be included within the scope of the input/output devices 308.

Also, the computing device 302 can include one or more network interface(s) 310. The network interface(s) 310 can be a point of interconnection between the computing device 302 and one or more networks 312. The network interface(s) 310 can be implemented in hardware, for example, as a network interface card (NIC), a network adapter, a LAN adapter or physical network interface. The network interface(s) 310 can be implemented in software. The network interface(s) 310 can be implemented as an expansion card or as part of a motherboard. The network interface(s) 310 can implement electronic circuitry to communicate using a specific physical layer and data link layer standard, such as Ethernet or Wi-Fi. The network interface(s) 310 can support wired and/or wireless communication. The network interface(s) 310 can provide a base for a full network protocol stack, allowing communication among groups of computers on the same local area network (LAN) and large-scale network communications through routable protocols, such as Internet Protocol (IP).

The one or more networks 312 can include any type of communications network, such as a local area network, a wide area network, a mesh network, an ad hoc network, a peer-to-peer network, the Internet, a cable network, a telephone network, a wired network, a wireless network, combinations thereof, and the like.

A device interface 314 can be part of the computing device 302 that provides hardware to establish communicative connections to other devices, such as a sequencer 316, a polynucleotide synthesizer 318, etc. The device interface 314 can also include software that supports the hardware. The device interface 314 can be implemented as a wired or wireless connection that does not cross a network. A wired connection may include one or more wires or cables physically connecting the computing device 302 to another device. The wired connection can be created by a headphone cable, a telephone cable, a SCSI cable, a USB cable, an Ethernet cable, FireWire, or the like. The wireless connection may be created by radio waves (e.g., any version of Bluetooth, ANT, Wi-Fi IEEE 802.11, etc.), infrared light, or the like.

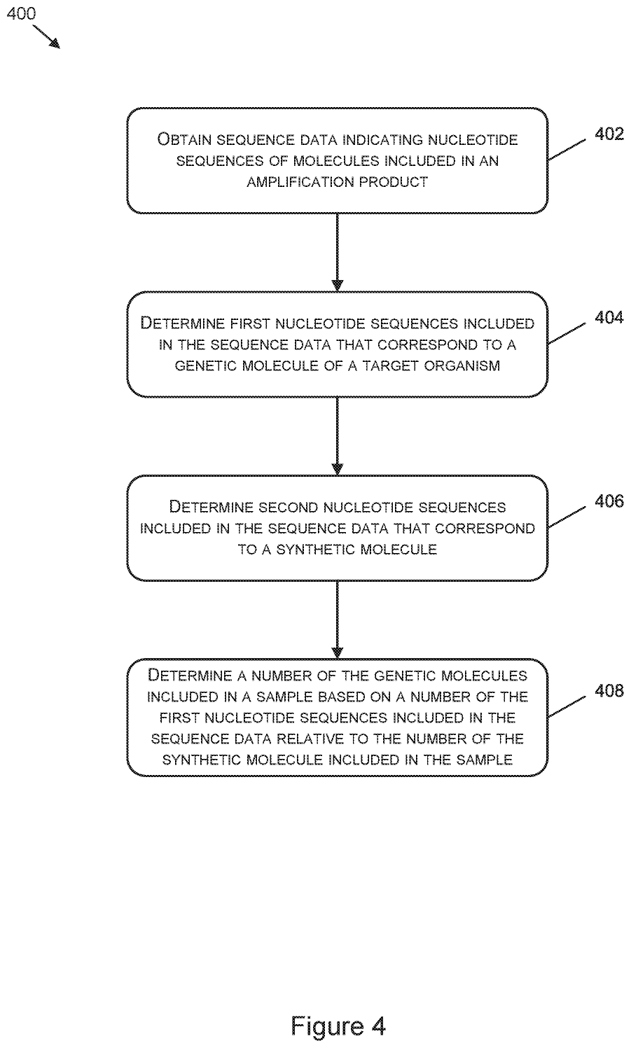

The computing device 302 can include multiple modules that may be implemented as instructions stored in the memory 306 for execution by processing unit(s) 304 and/or implemented, in whole or in part, by one or more hardware logic components or firmware. The memory 306 can be used to store any number of functional components that are executable by the one or more processors processing units 304. In many implementations, these functional components comprise instructions or programs that are executable by the one or more processing units 304 and that, when executed, implement operational logic for performing the operations attributed to the computing device 302. Functional components of the computing device 302 that can be executed on the one or more processing units 304 for implementing the various functions and features related quantification of molecules, as described herein, include target molecule quantification applications 320, such as synthetic molecule instructions 322, sequencing instructions 324, sequence analysis instructions 326, and target molecule quantification instructions 328. One or more of the sets of instructions, 322, 324, 326, 328 can be used to implement frameworks 100, 200, of FIG. 1 and FIG. 2 and the processes 400, 500, 600, 700 described with respect to FIGS. 4, 5, 6, and 7.

The synthetic molecule instructions 322 can be executable by the one or more processing units 304 to generate sequences of synthetic molecules that can be used to quantify a number of molecules included in a sample. In implementations, the synthetic molecule instructions 322 can generate some segments of nucleotide sequences of synthetic molecules using nucleotide sequences from biological organisms and some segments of the nucleotide sequences of synthetic molecules using computer-generated nucleotide sequences. In various implementations, the synthetic molecule instructions 322 can obtain nucleotide sequences from one or more bacteria and use the bacterial sequences to generate segments of a nucleotide sequence of the synthetic molecule. In illustrative examples, the synthetic molecule instructions 322 can add segments from ribosomal RNA genes, such as the 16S ribosomal RNA gene, to synthetic molecules. In implementations, the nucleotide sequences from biological organisms included in sequences of synthetic molecules can correspond to primers used in amplification operations. The synthetic molecule instructions 322 can also generate nucleotide sequences using pseudo-random or random number generator techniques. The synthetic molecule instructions 322 can assemble nucleotide sequences that include segments that have been generated using pseudo-random or random number generator techniques interspersed with segments obtained from nucleotide sequences of biological organisms in order to produce synthetic molecules that can be used in the quantification of molecules included in a sample.

The sequencing instructions 324 can be executable by the one or more processing units 304 to generate nucleotide sequences that correspond to given molecules. In example implementations, the sequencing instructions 324 can produce raw sequence data output that is sometimes referred to as reads. Each position in a read is an individual nucleotide determined by the sequencing instructions 324 based on properties of the nucleotides sensed by components of a machine, such as the sequencer 316. The properties sensed by the sequencer 316 can vary depending on the specific sequencing technology used. A read can represent a determination of which of the four nucleotides—A, G, C, and T (or U)—in a strand of DNA (or RNA) is present at a given position in the sequence.

The sequence analysis instructions 326 can be executable by the one or more processing units 304 to analyze sequence data produced by the sequencing instructions 324 and the sequencer 316. The reads included in the sequence data can be grouped together such that sequences having at least a threshold amount of identity can be grouped together as being associated with the same molecule. In example implementations, an amount of identity between sequences can be determined using a number of techniques, such as a Basic Alignment Search Tool (BLAST). In various implementations, an amount of sequence identity between molecules can be determined by performing a number of iterations of comparisons between nucleotides at various positions of the molecules. The comparisons between the nucleotides of the molecules can take place until each of the nucleotides of a first molecule have been compared with at least one of the nucleotides of a second molecule.

The sequence analysis instructions 326 can determine molecules corresponding to nucleotide sequences based on the presence of barcode sequences being detected in the nucleotide sequences. To illustrate, the sequence analysis instructions 326 can determine that a specified molecule can be identified based on a sub-sequence of nucleotides included in an overall nucleotide sequence for the molecule. In situations where the barcode sequence for a molecule matches a portion of a nucleotide sequence being analyzed, the sequence analysis instructions 326 can determine that the nucleotide sequence corresponds to the molecule associated with the barcode sequence. The sequence analysis instructions 326 can also group nucleotide sequences corresponding to the same molecule and determine a number of the nucleotide sequences that correspond to a specified molecule. In this way, the sequence analysis instructions 326 can determine a number of reads for each molecule present in a sample.

The target molecule quantification instructions 328 can be executable by the one or more processing units 304 to determine a number of target molecules included in a sample. In implementations, the target molecule quantification instructions 328 can determine a number of target molecules included in a sample based on a number of synthetic molecules included in the sample. In illustrative examples, the target molecule quantification instructions 328 can generate a ratio indicating a correlation between the number of reads included in the sequence data that correspond to a target molecule included in a sample and an initial number of the synthetic molecule included in the sample. In an example illustrative implementation, the target molecule quantification instructions 328 can utilize the following formula to determine an initial number of a target molecule included in a sample:

((t−s)/s)*n*v

where t is the total number of sequence reads for a sample, s is the number of sequence reads belonging to the synthetic spike-in, n is the known number of copies of spike-in added to the reaction, and v is the volume of sample added to the reaction.

After determining a number of target molecules included in a sample, in various implementations, a biological organism can be identified that is associated with the nucleotide sequence for a given target molecule. In implementations, a library of nucleotide sequences can include individual nucleotide sequences that correspond to individual biological organisms. The nucleotide sequence of a target molecule can be compared to the nucleotide sequences included in the library. In situations where a nucleotide sequence of a target molecule has at least a threshold amount of identity with respect to a nucleotide sequence included in the library, the target molecule can be identified as being associated with the biological organism that corresponds to the sequence in the library.

FIG. 4 is a flow diagram illustrating an example process 400 to quantify molecules included in a sample according to some implementations. At operation 402, the process 400 can include obtaining sequence data indicating nucleotide sequences of molecules included in an amplification product. The sequence data can be obtained from a sequencing machine that generates nucleotide sequences of molecules included in a sample. The amplification product can be produced using an amplification process, such as PCR or multiple displacement amplification.

The process 400 can also include, at operation 404, determining first nucleotide sequences included in the sequence data that correspond to a genetic molecule of a target organism. In various implementations, the nucleotides sequences that correspond to the genetic molecule can be identified based on a barcode sequence that corresponds to the genetic molecule. In illustrative examples, the genetic molecule can correspond to DNA of the target organism.

In addition, at operation 406, the process 400 can include determining second nucleotide sequences included in the sequence data that correspond to the synthetic molecule. The nucleotide sequences included in the sequence data can be compared against a nucleotide sequence of the synthetic molecule. Nucleotide sequences included in the sequence data having at least a threshold amount of identity with the sequence of the known synthetic molecule can be determined to correspond to the synthetic molecule.

At operation 408, the process 400 can include determining a number of the genetic molecules included in a sample based on a number of the first nucleotide sequences included in the sequence data relative to the number of the synthetic molecule included in the sample. That is, the number of reads corresponding to the target organism can be used with the initial number of synthetic molecules included in an original sample to determine the initial number of target molecules included in the sample. In implementations, the number of reads of the synthetic molecule included in the sequence data can also be used to quantify the initial number of target molecules included in the original sample. For example, a number between the total number of reads included in the sequence data and the number of reads included in the sequence data corresponding to the synthetic molecule can be used to estimate a number of reads corresponding to the target molecule. A ratio of the number of reads corresponding to the target molecule in the sequence data with respect to the initial number of synthetic molecules included in a sample can then be calculated to determine a number of target molecules included in the original sample.

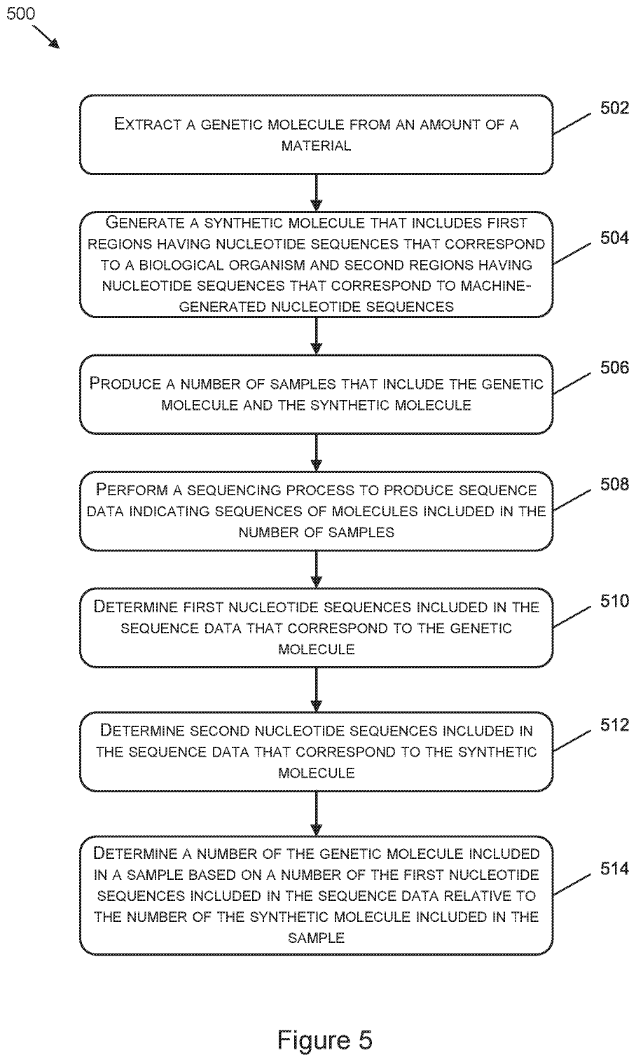

FIG. 5 is a flow diagram illustrating another example of a process 500 to quantify molecules included in a sample according to some implementations. At operation 502, the process 500 can include extracting a genetic molecule from an amount of material. In implementations, the amount of material can be obtained from an environment. In example implementations, the amount of material can be obtained from a solid material, a liquid material, or a gaseous material. In illustrative examples, the amount of material can be obtained from rock, a liquid hydrocarbon reservoir, human skin, soil, a food product, and the like. In various examples, the genetic material can be extracted from a cell included in the amount of material. In one or more additional examples, the genetic material can be extracted from a virus. In further examples, the genetic material can include free nucleic acids included in the amount of material.

At operation 504, the process 500 can include generating a synthetic molecule that includes first regions having nucleotide sequences that correspond to a biological organism and second regions having nucleotides that correspond to machine-generated nucleotide sequences. The machine-generated nucleotide sequences can also be referred to herein as synthetic nucleotide sequences. In example implementations, the synthetic molecule can be selected such that that 1) it will behave similarly to naturally-occurring target molecules during PCR, and 2) it can be easily distinguished from naturally-occurring target molecules. In principle, naturally-occurring target molecules absolutely known not to occur in the target samples could be used for the method described herein; however, use of such naturally-occurring targets (e.g., gene regions extracted from a bacterial species) would limit use to only those sample types that were already well-characterized. Therefore, instead, in some implementations, a synthetic DNA molecule is used as a spike-in. This synthetic molecule contains one or more regions of a naturally-occurring DNA sequence corresponding to the PCR priming sites being targeted for sequencing, interspersed by regions of DNA sequence unlikely to occur in nature. Because PCR-based DNA sequencing relies on the conserved priming sites to amplify target regions of interest, the naturally-occurring regions of the synthetic spike-in allow it to behave similarly to natural target molecules during PCR, while the non-natural sequences between priming regions permit it to be reliably distinguished from naturally-occurring molecules.

The process 500 can also include, at operation 506, producing a number of samples that include the genetic molecule and the synthetic molecule. For example, a quantity of the chosen spike-in molecule is obtained at a known concentration. The synthetic molecule can be added to the reagents to be used for initial PCR of the sample DNA such that the number of molecules of spike-in present in each PCR reaction can be accurately estimated. For example, in a typical PCR-base sequencing experiment, an experimenter might use ten PCR reactions of 50 μL volume, each comprising 40 μL PCR reagents and 10 μL unknown DNA sample that corresponds to the target molecule, one for each of ten unknown samples. Prior to adding the unknown DNA samples, the experimenter would typically create a ‘master mix’ of PCR reagents (e.g., totaling approximately 400 μL volume), which would then be split into multiple (e.g., ten) separate reactions. In example implementations, a volume of spike-in would be added to the ‘master mix’ such that the final concentration of spike-in in the master mix was precise (e.g., precisely 1000 copies per 40 μL). Thus, it would be known that in the sequencing PCR amplification, each sample would contain a precise number (e.g., 1000) of synthetic molecules alongside an unknown quantity of naturally-occurring target molecules.

Additionally, at operation 508, the process 500 can include performing a sequencing process to produce sequence data indicating sequences of molecules included in the number of samples. In example implementations, the sequencing process can be performed after an amplification process, such as PCR, is performed with respect to the samples. The sequence data can include nucleotide sequences of a number of molecules included in the samples, such as the synthetic molecule, one or more target molecules, and one or more contaminant molecules.

Further, the process 500 can, at operation 510, include determining first nucleotide sequences included in the sequence data that correspond to the genetic molecule and, at operation 512, the process 500 can include determining second nucleotide sequences included in the sequence data that correspond to the synthetic molecule. In this way, the number of reads attributed to the genetic molecule can be determined as well as the number of reads attributed to the target molecule.

At operation 514, the process 500 can include determining a number of the genetic molecule included in a sample based on a number of the first nucleotide sequences included in the sequence data relative to the number of the synthetic molecule included in the sample. In implementations, the total number of reads included in the sequence data per sample may not be well-correlated to the starting quantity of molecules in the sample. However, because the starting quantity of synthetic molecules is known, the starting concentration of naturally-occurring target molecules can be estimated. In example implementations, the following formula is used:

((t−s)/s)*n*v

where t is the total number of sequence reads for a sample, s is the number of sequence reads belonging to the synthetic spike-in, n is the known number of copies of spike-in added to the reaction, and v is the volume of sample added to the reaction.

The information derived from quantification using spike-in synthetic molecules can be used for operations requiring more precise knowledge of the starting quantity of target molecules than is provided by conventional techniques. For example, a common problem in PCR-based target molecule sequencing is the presence of contaminant molecules in the reagents themselves. These contaminants can themselves be considered as spike-in molecules of unknown concentration and identity. But because they are derived from reagents and not samples, they are present at a similar starting copy number in each PCR reaction, and can be readily identified by means of correlation with the synthetic molecule: unique sequence types that are found in similar sequence read abundance ratios compared to the synthetic molecule sequences reads across many samples can be identified as reagent-based contaminants, and excluded from subsequent analysis.

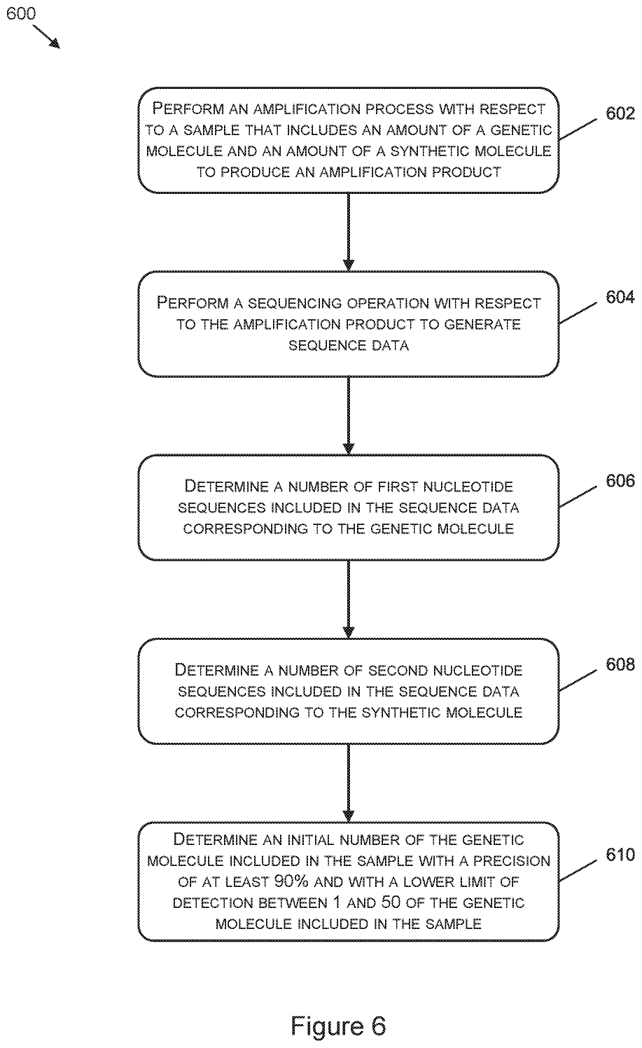

FIG. 6 is a flow diagram illustrating an example process 600 to quantify molecules included in a sample with specified limits of detection according to some implementations. At operation 602, the process 600 can include performing an amplification process with respect to a sample that includes an amount of a genetic molecule and an amount of a synthetic molecule to produce an amplification product. The amplification process can include a PCR reaction in some situations. In addition, the amplification process can include a multiple displacement amplification process. In example implementation, the amplification process may not include a PCR process that does not include real-time PCR process, a quantitative PCR process, or a droplet digital PCR process.

At operation 604, the process 600 can include performing a sequencing operation with respect to the amplification product to generate sequence data. At operation 606, the sequence data can be used to determine a number of first nucleotide sequences that correspond to the genetic molecule and, at 608, the sequence data can be used to determine a number of second nucleotide sequences corresponding to the synthetic molecule.

The process 600 can also include, at operation 610, determining an initial number of the genetic molecule included in the sample with a precision of at least 90% and with a lower limit of detection between 1 and 50 of the genetic molecule included in the sample. In various implementations, the precision can be at least 90%, at least 92%, at least 95%, at least 98%, or at least 99%. In addition, the lower limits of detection can be between 1 and 25 molecules or between 1 and molecules.

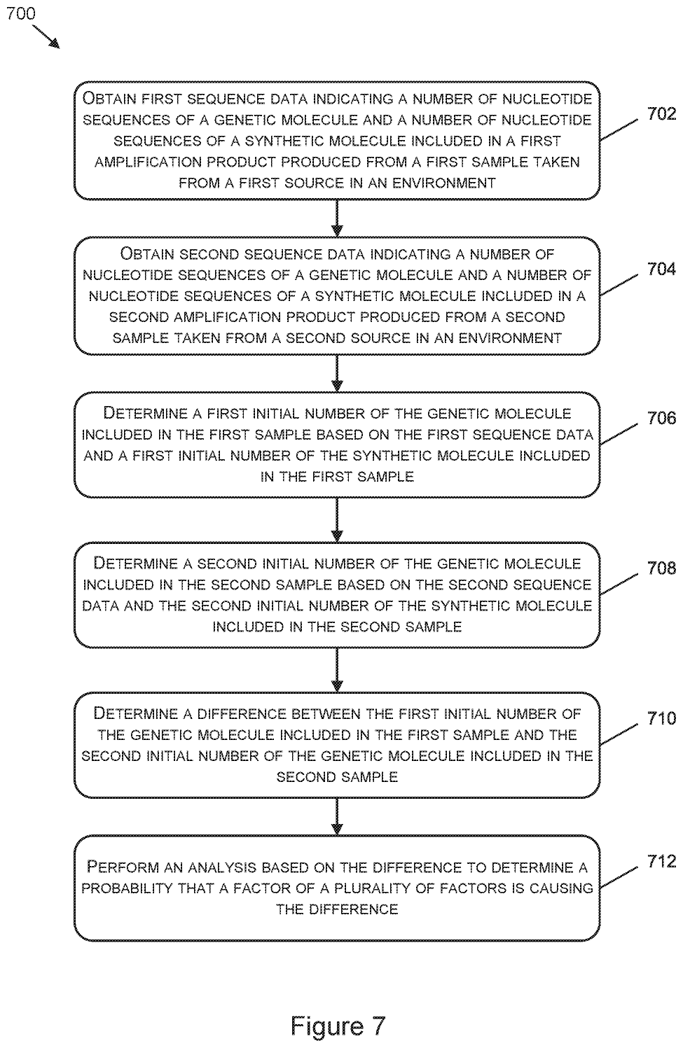

FIG. 7 is a flow diagram illustrating an example process 700 to quantify molecules included in samples obtained from multiple sources according to some implementations. At operation 702, the process 700 can include obtaining first sequence data indicating a number of nucleotides sequences of a genetic molecule and a number of nucleotide sequences of a synthetic molecule included in a first amplification product. The first amplification product can be produced from a first sample taken from a first source in an environment.

At operation 704, the process 700 can include obtaining second sequence data indicating a number of nucleotide sequences of a genetic molecule and a number of sequences of the synthetic molecule included in a second amplification product. The second amplification product can be produced from a second sample taken from a second source in the environment. In an illustrative example, the first source can include a fossil-fuel based petroleum substance or a natural gas substance and the second source can include a rock-based substance. In other illustrative examples, the first source can include a first portion of a body of an individual and the second source can include a second portion of the body of the individual.

The process 700 can also include, at operation 706, determining a first initial number of the genetic molecule included in the first sample based on the first sequence data and a first initial number of the synthetic molecule included in the first sample. In addition, at operation 708, the process 700 can include determining a second initial number of the genetic molecule included in the second sample based on the second sequence data and the second initial number of the synthetic molecule included in the second sample. In implementations, the first initial number of the genetic molecule included in the first sample and the second initial number of the genetic molecule included in the second sample can be based on a number of reads corresponding to the genetic molecule included in the sequence data for a first amplification product derived from the first sample and a number of reads corresponding to the genetic molecule included in the sequence data for a second amplification product derived from the second sample.

Additionally, at operation 710, the process 700 can include determining a difference between the first initial number of the genetic molecule included in the first sample and the second initial number of the genetic molecule included in the second sample. Further, at operation 712, the process 700 can include performing an analysis based on the difference to determine a probability of a factor of a plurality of factors causing the difference. That is, an analysis can be performed to determine one or more possible confounding factors that can be causing the difference between the initial number of the genetic molecule included in samples taken from different sources in the environment. In implementations, the difference can be caused by a contaminant in the environment. In additional implementations, the difference can be caused by other factors, such as temperature, humidity, and/or pressure differences. Various statistical techniques can be used to identify contaminant molecules in samples. In an illustrative example, Spearman's correlation can be used to identify a contaminant in a sample.

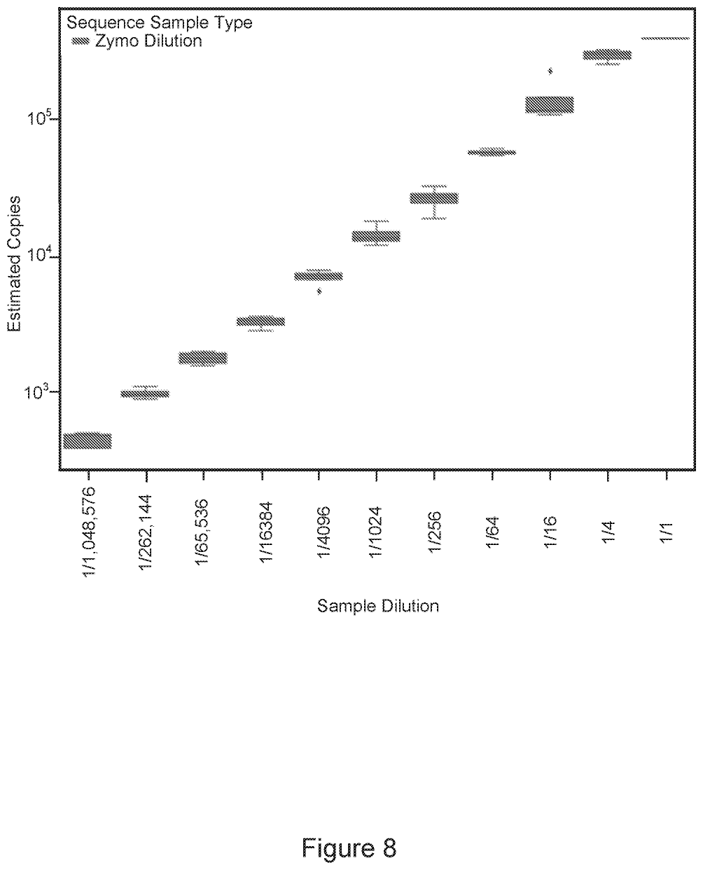

FIG. 8 is a visual representation of the process of adding synthetic spike-in molecules to raw samples to generate more accurate assessments of their quantities in the samples relative to conventional techniques. In FIG. 8, the x-axis indicates a dilution factor for a sample and the y-axis indicates the number of reads for molecules included in an amplification product.

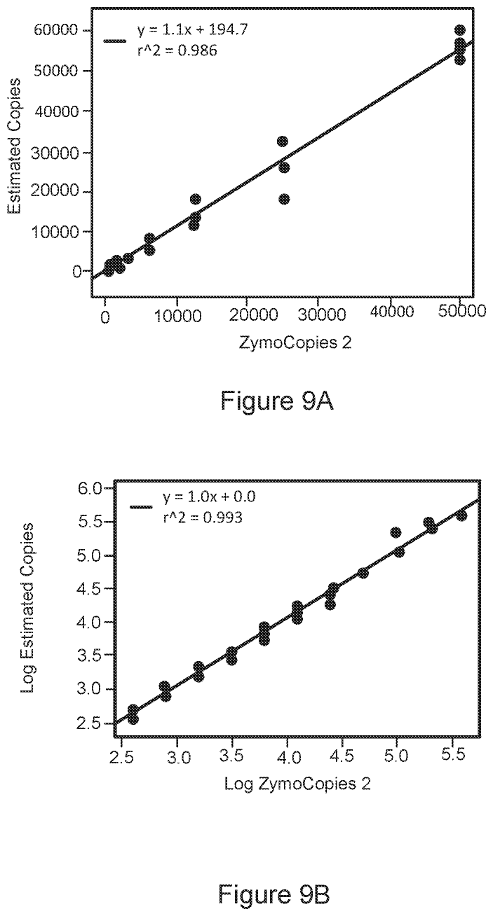

As shown in FIGS. 9A and 9B, in example implementations, experiments diluting a commercially available microbial mock community (e.g., “ZymoMock”; Zymo Research) demonstrate a linear response in the estimated absolute copy number derived from a single spike-in of 1000 copies of synthetic SSU-rRNA per reaction, with clear separation between 2-fold dilution levels across the range tested. The estimated copies of the y-axis are determined using techniques and implementations described herein. As shown in FIGS. 9A and 9B, in example implementations, these estimates correspond well to expected ZymoMock inputer values across three orders of magnitude, with homoskedastic variance of estimates in log-transformed space.

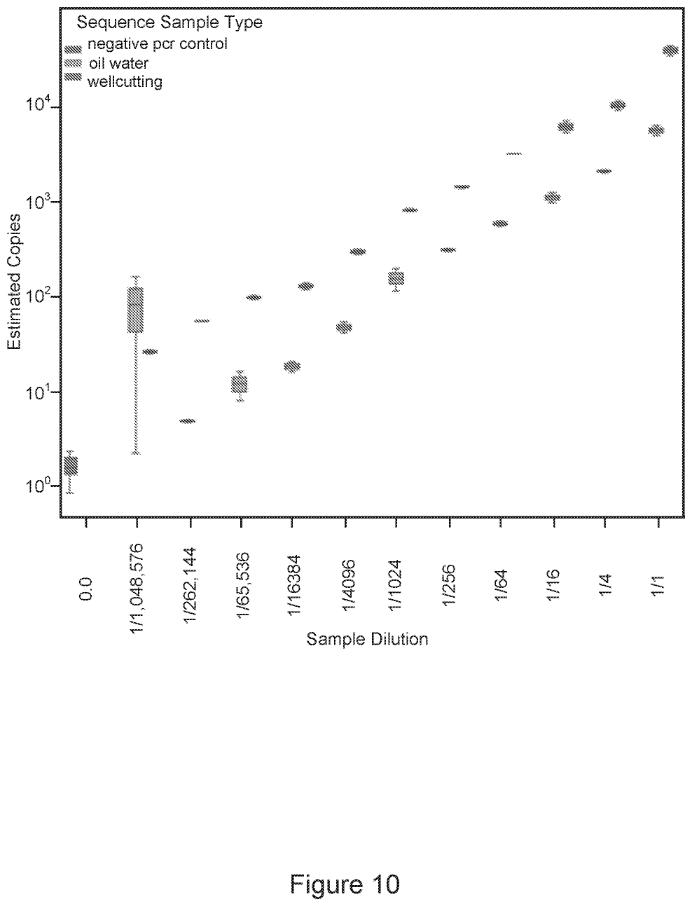

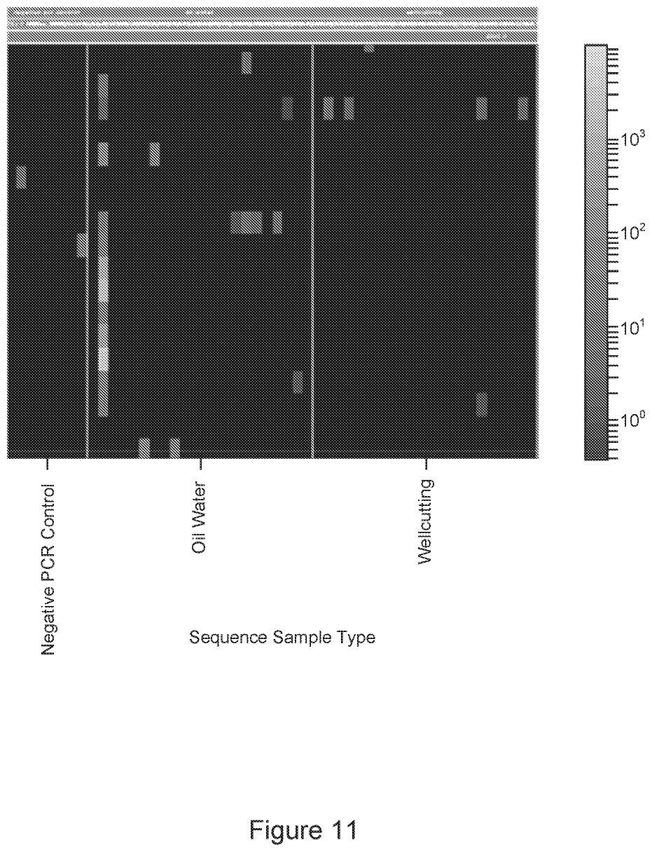

As shown in FIG. 10, in example implementations, estimates of absolute starting copy number in low-abundance natural subsurface communities show linear response in estimates down to very high dilutions, with duplicate measurements of two sample types showing precise estimates down to values around a single copy per μL in the starting DNA extraction. Furthermore, these values are also distinguishable from eight replicate measurements of the no-template control, which was estimated at 1.56 copies/μL (sd=0.46). FIG. 11 is a chart showing how the outlier abundance measurement in FIG. 10 can be interpreted via the specific sequence types observed in the outlier sample and compared to available databases highlighting the utility over qPCR and other methods where the sequence information is not known.

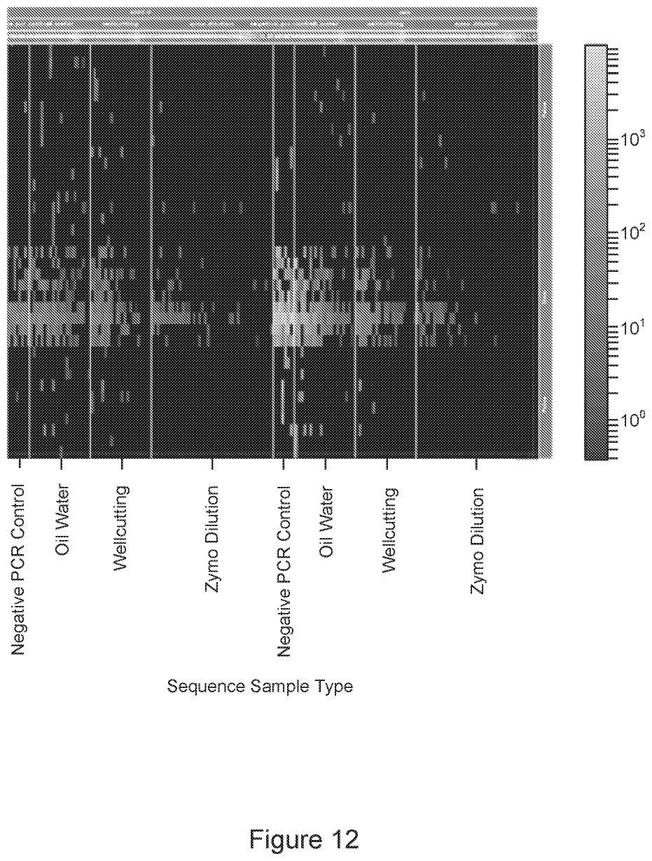

Furthermore, as shown in FIG. 12, in example implementations, the fact that quantification is done simultaneously with community sequencing means that more information can be gleaned from outlier samples relative to conventional techniques. For example, one replicate of the lowest dilution of the ‘oil-water’ sample above was much higher than expected given the dilution series. This sample was found to have several unique microbial sequences that were not present in either the replicate dilution or any other replicates of the sample:

In example implementations, by querying these sequences against a public database, it is possible to conclude, for example, that the bacteria represented by these sequences are common members of the human skin microbiome, and thus conjecture that the underlying reason for the outlier copy number estimate was due to the chance contamination of that single well. This represents a powerful improvement over purely quantitative methods such as qPCR or ddPCR.

The ability to simultaneously glean absolute abundance information and community sequence data also allows the methods disclosed herein to be used to distinguish reagent contaminant sequences from the original, sample-derived sequences. Because both the synthetic spike-in sequences and unknown reagent contaminants are present at a constant absolute abundance in initial samples, while sequences from samples vary according to sample starting concentration and natural community variation, correlation in relative abundance with the known spike-in sequence can be used to identify reagent contaminants.

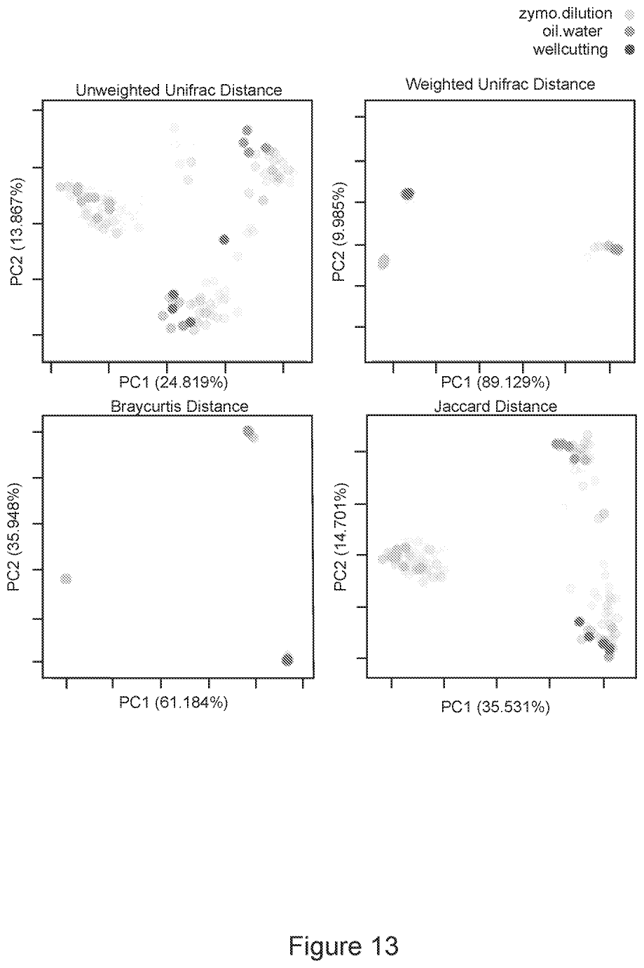

As shown in FIG. 13, in example implementations, Spearman rank correlation or other statistical approaches may reveal a set of these putative contaminant sequences, which (also consistent with being contaminants) are present across all sample types, and are especially abundant in no-template control “blank” samples.

Identifying reagent contaminants in this way also allows for estimates of community diversity to be corrected for contamination, which can be a problem in low-biomass samples, and can lead to spurious findings. As shown in FIG. 13, in example implementations, principal coordinate analysis ordination of sample similarity measures may show that the highly-diluted samples (e.g., ‘low biomass’ samples) from different original sample types tend to be more similar to one another, as the shared reagent contaminant sequences makes up increasingly larger proportions of each sample.

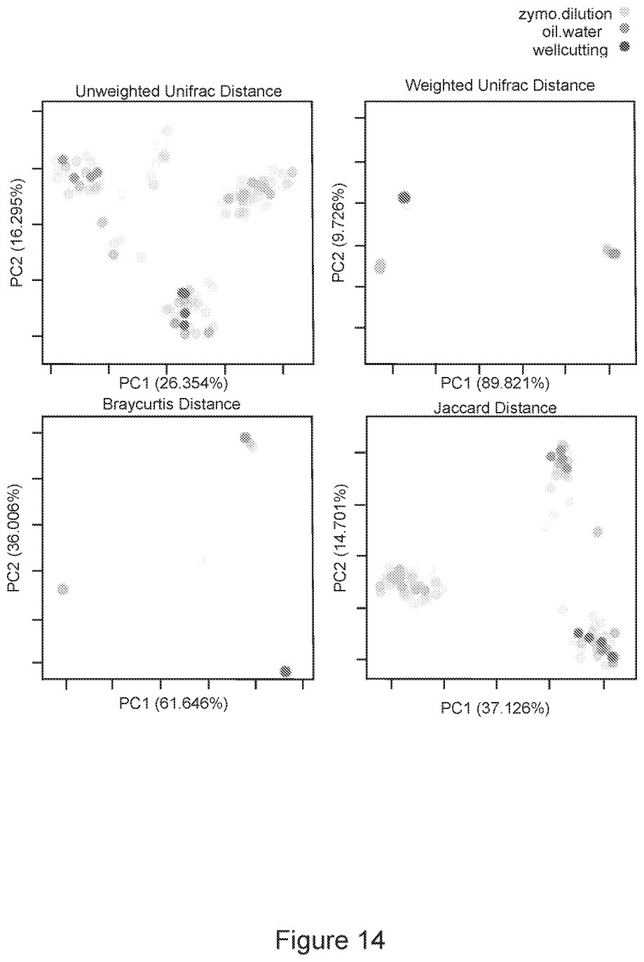

As shown in FIG. 14, after removing reagent contaminants detected with the disclosed spike-in sequences, this trend may be largely eliminated. FIG. 14 is a chart showing how, after removing reagent contaminants detected with the disclosed spike-in sequences, a trend of low biomass samples tending to be more similar to one another as the shared reagent contaminant sequences made up increasingly larger proportions of the samples may be largely eliminated.

Thus, the disclosed method of using single-concentration synthetic spike-in molecules improves on existing methods for universal bacterial quantitation of low-biomass samples by, for example, yielding estimates of SSU-rRNA copy number that are precise down to levels that meet or improve upon existing methods like ddPCR; using very inexpensive reagents that are compatible with any sample type, unlike spike-ins derived from naturally-occurring DNA sequences; and requiring minimal additional labor or analytical complexity, and requiring no additional equipment.

Certain implementations are described herein as including logic or a number of components, modules, or mechanisms. Modules may constitute either software modules (e.g., code embodied on a machine-readable medium or in a transmission signal) or hardware modules. A hardware module is a tangible unit capable of performing certain operations and may be configured or arranged in a certain manner. In example implementations, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various implementations, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired) or temporarily configured (e.g., programmed) to operate in a certain manner and/or to perform certain operations described herein. Considering implementations in which hardware modules are temporarily configured (e.g., programmed), each of the hardware modules need not be configured or instantiated at any one instance in time. For example, where the hardware modules comprise a computer processor that is specially configured (e.g., using software), the computer processor may be specially configured as respective different hardware modules at different times. Software may accordingly configure a processor, for example, to constitute a specified hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In implementations in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices and can operate on a resource (e.g., a collection of information).

The various operations of example processes described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules and instructions referred to herein may, in some example implementations, comprise processor-implemented modules.

Similarly, the processes described herein may be at least partially processor-implemented. For example, at least some of the operations of a process may be performed by one or more processors or processor-implemented modules. The performance of certain of the operations may be distributed among the one or more processors, not only residing within a single machine, but deployed across a number of machines. In some example implementations, the processor or processors may be located in a single location (e.g., within a home environment, an office environment or as a server farm), while in other implementations the processors may be distributed across a number of locations.

The one or more processors may also operate to support performance of the relevant operations in a “cloud computing” environment or as a “software as a service” (SaaS). For example, at least some of the operations may be performed by a group of computers (as examples of machines including processors), these operations being accessible via a network (e.g., the network 312 of FIG. 3) and via one or more appropriate interfaces (e.g., APIs).

Example implementations may be implemented in digital electronic circuitry, or in computer hardware, firmware, software, or in combinations of them. Example implementations may be implemented using a computer program product, e.g., a computer program tangibly embodied in an information carrier, e.g., in a machine-readable medium for execution by, or to control the operation of, data processing apparatus, e.g., a programmable processor, a computer, or multiple computers.

A computer program can be written in any form of programming language, including compiled or interpreted languages, and it can be deployed in any form, including as a stand-alone program or as a module, subroutine, or other unit suitable for use in a computing environment. A computer program can be deployed to be executed on one computer or on multiple computers at one site or distributed across multiple sites and interconnected by a communication network.

In example implementations, operations may be performed by one or more programmable processors executing a computer program to perform functions by operating on input data and generating output. Method operations can also be performed by, and apparatus of example implementations may be implemented as, special purpose logic circuitry (e.g., a FPGA or an ASIC).

The computing system can include clients and servers. A client and server are generally remote from each other and typically interact through a communication network. The relationship of client and server arises by virtue of computer programs running on the respective computers and having a client-server relationship to each other. In implementations deploying a programmable computing system, it will be appreciated that both hardware and software architectures require consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware may be a design choice.

Although the features herein have been described with reference to specific example implementations, it will be evident that various modifications and changes may be made to these implementations without departing from the broader spirit and scope of the present disclosure. Accordingly, the specification and drawings are to be regarded in an illustrative rather than a restrictive sense. The accompanying drawings that form a part hereof, show by way of illustration, and not of limitation, specific implementations in which the subject matter may be practiced. The example implementations illustrated are described in sufficient detail to enable those skilled in the art to practice the teachings disclosed herein. Other implementations may be utilized and derived therefrom, such that structural and logical substitutions and changes may be made without departing from the scope of this disclosure. This Detailed Description, therefore, is not to be taken in a limiting sense, and the scope of various implementations is defined only by the appended claims, along with the full range of equivalents to which such claims are entitled.

### Examples

Example 1. A method comprising: obtaining an amount of a material from a source; extracting a genetic molecule from a cell included in the amount of material, the genetic molecule having a first sequence of nucleotides; generating, by one or more computing devices, first data indicating one or more sequences of nucleotides; generating, by at least one computing device of the one or more computing devices, second data indicating a second sequence of nucleotides for a synthetic molecule, the synthetic molecule including first regions that include nucleotide sequences of a biological organism and second regions that include additional nucleotide sequences selected from the one or more sequences of nucleotides included in the first data; producing a volume of a mixture that includes a first portion having an amount of the synthetic molecule and a second portion that includes one or more amplification reagents; producing a plurality of samples from the mixture, individual samples of the plurality of samples including a portion of the volume of the mixture, and an additional portion that includes an amount of the genetic molecule; performing an amplification process to produce an amplification product for a sample of the plurality of samples, wherein the amplification product includes an amplified number of the genetic molecule and an amplified number of the synthetic molecule that is greater than an initial number and an initial number of the synthetic molecule included in the sample; performing a sequencing process to determine nucleotide sequences of molecules included in the amplification product; obtaining, by at least one computing device of the one or more computing devices and based on the sequencing process, sequence data indicating the nucleotide sequences of the molecules included in the amplification product; determining, by at least one computing device of the one or more computing devices and based on the sequence data, a first number of nucleotide sequences included in the sequence data that correspond to the genetic molecule and a second number of nucleotide sequences included in the sequence data that correspond to the synthetic molecule; and determining, by at least one computing device of the one or more computing devices, the initial number of the genetic molecule included in the sample based at least partly on a number of the synthetic molecule included in the sample, a volume of the sample, and the first number of nucleotide sequences included in the sequence data relative to the second number of nucleotide sequences included in the sequence data.

Example 2. The method of example 1, wherein: the amplification process includes a polymerase chain reaction (PCR) or multiple displacement amplification (MDA) technique; the nucleotide sequences of the biological organism correspond to primers included in the one or more amplification reagents; and the nucleotide sequences of the biological organism are selected from conserved regions of the ribosomal ribonucleic acid gene (rRNA gene).

Example 3. The method of example 1 or 2, comprising implementing one or more pseudo-random number generators to generate the first data indicating the one or more sequences of nucleotides.

Example 4. The method of example 3, comprising dividing a nucleotide sequence generated using the one or more pseudo-random number generators into a plurality of segments to generate a plurality of sequences of nucleotides included in the first data.

Example 5. The method of any of examples 1-4, wherein the volume of the mixture includes a third portion that includes an amount of an additional molecule, and the method comprises: determining, by at least one computing device of the one or more computing devices and based on the sequence data, a third number of nucleotide sequences included in the sequence data that correspond to the additional molecule; and determining, by at least one computing device of the one or more computing devices, an initial number of the additional molecule included in the sample based at least partly on the number of the synthetic molecule included in the sample, the volume of the sample, and the third number of nucleotide sequences included in the sequence data relative to the second number of nucleotide sequences included in the sequence data.

Example 6. The method of example 5, comprising: determining, by at least one computing device of the one or more computing devices, a correlation between the number of the synthetic molecule included in the sample and the initial number of an additional molecule included in the sample; determining, by at least one computing device of the one or more computing devices, that the correlation satisfies one or more threshold criteria; determining, by at least one computing device of the one or more computing devices, that the additional molecule is a contaminant included in the one or more amplification reagents.

Example 7. The method of example 5, comprising: determining, by at least one computing device of the one or more computing devices, that the third number of the additional molecule is greater than a threshold number; and determining, by at least one computing device of the one or more computing devices, that the additional molecule is another genetic molecule included in the sample.

Example 8. The method of any of examples 1-7, comprising determining the number of the synthetic molecule included in the sample based on a number of samples derived from the volume of the mixture and the amount of the synthetic molecule included in the volume of the mixture.

Example 9. The method of any of examples 1-8, wherein the genetic molecule includes deoxyribonucleic acid (DNA) or ribonucleic acid (RNA) shared with additional biological organisms.

Example 10. A system comprising: at least one hardware processor; and a computer-readable medium storing instructions that, when executed by the at least one hardware processor, cause the at least one hardware processor to perform operations comprising: obtaining sequence data indicating nucleotide sequences of molecules included in an amplification product, the amplification product corresponding to a sample that has undergone an amplification process to increase an initial number of molecules included in the sample: determining, based on the sequence data, a first number of nucleotide sequences included in the sequence data that correspond to a genetic molecule included in the sample and a second number of nucleotide sequences included in the sequence data that correspond to a synthetic molecule included in the sample, the synthetic molecule including first regions that include first nucleotide sequences of the genetic molecule and second regions that include second nucleotide sequences selected from one or more machine-generated sequences of nucleotides; and determining, a number of the genetic molecule included in the sample based at least partly on a number of the synthetic molecule included in the sample, a volume of the sample, and the first number of nucleotide sequences included in the sequence data relative to the second number of nucleotide sequences included in the sequence data.

Example 11. The system of example 10, wherein the computer-readable medium stores additional instructions that, when executed by the at least one hardware processor, cause the at least one hardware processor to perform additional operations comprising: performing a comparison of a nucleotide sequence of the genetic molecule to one or more nucleotide sequences included in a library of nucleotide sequences, the library of nucleotide sequences including a plurality of nucleotide sequences that correspond to a plurality of additional biological organisms; and determining, based on the comparison, an additional biological organism of the plurality of additional biological organisms that corresponds to the genetic molecule.

Example 12. The system of example 11, wherein the computer-readable medium stores additional instructions that, when executed by the at least one hardware processor, cause the at least one hardware processor to perform additional operations comprising: determining, based on the sequence data, a third number of nucleotide sequences included in the sequence data that correspond to an additional genetic molecule included in the sample; performing a comparison of an additional nucleotide sequence of the additional genetic molecule to the one or more nucleotide sequences included in the library of nucleotide sequences; determining, based on the comparison, a second additional biological organism of the plurality of additional biological organisms that corresponds to the additional genetic molecule; and determining that the additional biological organism and the second additional biological organism are present in environments where at least one of crude oil, natural gas, and formation water are located.

Example 13. The system of any of examples 10-12, wherein the computer-readable medium stores further instructions that, when executed by the at least one hardware processor, cause the at least one hardware processor to perform further operations comprising: determining, based on the sequence data, a third number of nucleotide sequences included in the sequence data that correspond to an additional molecule; determining a correlation between the third number of nucleotide sequences and the second number of nucleotide sequences; determining that the correlation satisfies one or more threshold criteria; and determining that the additional molecule is a contaminant included in the one or more amplification reagents.

Example 14. The system of any of examples 10-13, comprising a device interface to couple to a sequencing machine; and wherein the sequence data is obtained from the sequencing machine.

Example 15. A method comprising: obtaining, by one or more computing devices, sequence data indicating nucleotide sequences of molecules included in an amplification product, the amplification product corresponding to a sample that has undergone an amplification process to increase an initial number of molecules included in the sample; determining, by at least one computing device of the one or more computing devices and based on the sequence data, a first number of nucleotide sequences included in the sequence data that correspond to a genetic molecule included in the amplification product and a second number of nucleotide sequences included in the sequence data that correspond to a synthetic molecule included in the amplification product, the synthetic molecule including first regions that include nucleotide sequences of a biological organism and second regions that include nucleotide sequences selected from one or more machine-generated sequences of nucleotides; generating a function to determine a number of the genetic molecule included in the sample; and determining, by at least one computing device of the one or more computing devices, the number of the genetic molecule included in the sample based on the number of the synthetic molecule included in the sample and the first number of nucleotide sequences included in the sequence data.

Example 16. The method of example 15, comprising: determining, based on a presence of the genetic molecule in the sample, that one or more biochemical reactions have taken place in an environment from which the sample was obtained.

Example 17. The method of example 16, wherein the one or more biochemical reactions include at least one of a nitrate reducing reaction, a sulfate reducing reaction, methanogenesis, a hydrocarbon conversion reaction, or a biosurfactant generating reaction.

Example 18. The method of any of examples 15-17, comprising: extracting the genetic molecule from a cell included in an amount of a fluid obtained from a subterranean environment that stores at least one of a fossil fuel-based petroleum substance or natural gas.

Example 19. The method of any of examples 15-18, comprising: performing comparisons between a nucleotide sequence of the genetic molecule with respect to a plurality of additional nucleotide sequences that are associated with a plurality of individuals; determining, based on the comparisons, a threshold amount of identity between the nucleotide sequence of the genetic molecule and an additional nucleotide sequence of the plurality of additional nucleotide sequences; and identifying an individual of the plurality of individuals that corresponds to the additional nucleotide sequence.

Example 20. The method of any of examples 15-19, comprising: obtaining an amount of a material from an environment; extracting a cell from the material that includes the genetic molecule; performing comparisons between a nucleotide sequence of the genetic molecule with respect to a plurality of additional nucleotide sequences that are associated with contaminants; determining, based on the comparisons, a threshold amount of identity between the nucleotide sequence of the genetic molecule and an additional nucleotide sequence of the plurality of additional nucleotide sequences; and identifying a contaminant of the environment, wherein the contaminant corresponds to the additional nucleotide sequence.

Example 21. The method of any of examples 15-20, wherein the number of the genetic molecule included in the sample is determined with a precision of at least 95% and with a lower limit of detection between 1 and 10 of the genetic molecule being included in the sample.

Example 22. The method of any of examples 15-21, comprising: identifying a first barcode sequence included in a first nucleotide sequence; determining, based on the first barcode sequence, that the first nucleotide sequence corresponds to a first molecule; identifying a second barcode sequence included in a second nucleotide sequence; and determining, based on the second barcode sequence, that the second nucleotide sequence corresponds to a second molecule.

Example 23. The method of example 22, comprising: producing a first group of nucleotide sequences that include the first barcode sequence; producing a second group of nucleotide sequences that include the second barcode sequence; determining the number of the first nucleotide sequences based on a number of nucleotide sequences included in the first group; and determining the number of the second nucleotide sequences based on a number of nucleotide sequences included in the second group.

Example 24. A method comprising: obtaining an amount of a material from a source, the material including a genetic molecule; producing a sample having a volume from 10 microliters to 500 microliters and that includes (i) at least a portion of the amount of the material, (ii) one or more amplification reagents, and (iii) an amount of a synthetic molecule, the synthetic molecule including first regions that correspond to primers included in one or more amplification reagents and second regions that include computer-generated nucleotide sequences; performing an amplification process to produce an amplification product for the sample, wherein the amplification product includes an amplified number of the genetic molecule that is greater than an initial number of the genetic molecule included in the sample and an amplified number of the synthetic molecule that is greater than an initial number of the synthetic molecule included in the sample; performing a sequencing process to determine nucleotide sequences of molecules included in the amplification product; obtaining, by at least one computing device of one or more computing devices and based on the sequencing process, sequence data indicating a number of first nucleotide sequences corresponding to the genetic molecule and a number of second nucleotide sequences corresponding to the synthetic molecule; and determining, by at least one computing device of the one or more computing devices and based on the initial number of the synthetic molecule included in the sample, the initial number of the genetic molecule included in the sample with a precision of at least 95% and with a lower limit of detection between 1 and 10 of the genetic molecules included in the sample.

Example 25. The method of example 24, comprising: identifying a first barcode sequence included in a first nucleotide sequence; determining, based on the first barcode sequence, that the first nucleotide sequence corresponds to a first molecule; identifying a second barcode sequence included in a second nucleotide sequence; and determining, based on the second barcode sequence, that the second nucleotide sequence corresponds to a second molecule.

Example 26. The method of example 25, comprising: producing a first group of nucleotide sequences that include the first barcode sequence; producing a second group of nucleotide sequences that include the second barcode sequence; determining the number of the first nucleotide sequences based on a number of nucleotide sequences included in the first group; and determining the number of the second nucleotide sequences based on a number of nucleotide sequences included in the second group.

Example 27. The method of any of examples 24-26, comprising: generating, using a random number generator or a pseudo-random number generator, data indicating one or more sequences of nucleotides; and determining the computer-generated sequences using at least a portion of the one or more sequences of nucleotides.

Example 28. The method of any of examples 24-27, wherein: the amplification process includes a polymerase chain reaction (PCR) technique or a multiple displacement amplification (MDA) technique.

Example 29. The method of example 28, wherein the polymerase chain reaction does not include a real-time PCR technique or a quantitative PCR technique.

Example 30. The method of any of examples 24-29, wherein the sample includes an amount of an additional molecule, and the method comprises: determining, by at least one computing device of the one or more computing devices and based on the sequence data, a third number of nucleotide sequences included in the sequence data that correspond to the additional molecule; and determining, by at least one computing device of the one or more computing devices, the initial number of the additional molecule included in the sample based at least partly on the number of the synthetic molecule included in the sample, the volume of the sample, and the third number of nucleotide sequences included in the sequence data relative to the second number of nucleotide sequences of the synthetic molecule included in the sequence data.

Example 31. The method of example 30, comprising: determining, by at least one computing device of the one or more computing devices, a correlation between the initial number of the additional molecule included in the sample and the initial number of the synthetic molecule included in the sample; determining that the correlation satisfies one or more criteria; and determining, by at least one computing device of the one or more computing devices, that the additional molecule is a contaminant.

Example 32. A system comprising: at least one hardware processor; and a computer-readable medium storing instructions that, when executed by the at least one hardware processor, cause the at least one hardware processor to perform operations comprising: obtaining sequence data indicating a number of first nucleotide sequences corresponding to a first molecule and a number of second nucleotide sequences corresponding to a second molecule, wherein the sequence data is derived from an amplification product that corresponds to a sample that has undergone an amplification process to increase an initial number of the first molecule and an initial number of the second molecule included in the sample; determining that the first molecule corresponds to a genetic molecule of a biological organism and the second molecule corresponds to a synthetic molecule, the synthetic molecule including first regions that correspond to primers included in one or more amplification reagents of the amplification process and second regions that include computer-generated nucleotide sequences, and determining, based on the initial number of the second molecule included in the sample, the initial number of the first molecule included in the sample with a precision of at least 95% and with a lower limit of detection between 1 and 10 of the genetic molecules included in the sample.

Example 33. The system of example 30, wherein the computer-readable medium stores additional instructions that, when executed by the at least one hardware processor, cause the at least one hardware processor to perform additional operations comprising: determining a difference between the number of first nucleotide sequences and the number of second nucleotide sequences included in the sequence data; and determining a ratio corresponding to (i) a product of the initial number of the synthetic molecule included in the sample and the difference between the number of first nucleotide sequences and the number of second nucleotide sequences included in the sequence data with respect to (ii) the second number of nucleotide sequences included in the sequence data; and wherein the number of the genetic molecule included in the sample is determined based on the ratio.

Example 34. The system of example 32 or 33, wherein the computer-readable medium stores additional instructions that, when executed by the at least one hardware processor, cause the at least one hardware processor to perform additional operations comprising: identifying a barcode sequence included in a nucleotide sequence of the sequence data; determining, based on the barcode sequence, that the nucleotide sequence corresponds to the first molecule; and producing a modified nucleotide sequence by removing the barcode sequence from the nucleotide sequence.

Example 35. The system of example 34, wherein the computer-readable medium stores further instructions that, when executed by the at least one hardware processor, cause the at least one hardware processor to perform further operations comprising: performing a comparison of the modified nucleotide sequence to one or more nucleotide sequences included in a library of nucleotide sequences, the library of nucleotide sequences including a plurality of nucleotide sequences that correspond to a plurality of biological organisms; and determining, based on the comparison, that the modified nucleotide sequence corresponds to a nucleotide sequence of a bacteria included in the library of nucleotide sequences.

Example 36. The system of any of examples 32-35, wherein the sequence data is related to a sample obtained from a first source included in an environment and, wherein the computer-readable medium stores additional instructions that, when executed by the at least one hardware processor, cause the at least one hardware processor to perform additional operations comprising: obtaining additional sequence data indicating a number of first additional nucleotide sequences corresponding to the first molecule and a number of second additional nucleotide sequences corresponding to the second molecule, wherein the sequence data is derived from an additional amplification product that corresponds to an additional sample that has undergone an additional amplification process to increase an initial number of the first molecule and an initial number of the second molecule included in the additional sample, the additional sample being obtained from a second source included in the environment; determining, based on the number of second additional nucleotide sequences, the initial number of the first molecule included in the additional sample; and determining a difference between the initial number of the first molecule included in the sample and the initial number of the first molecule included in the additional sample.

Example 37. The system of example 36, wherein the computer-readable medium stores further instructions that, when executed by the at least one hardware processor, cause the at least one hardware processor to perform further operations comprising: determining that the difference between the initial number of the first molecule included in the sample and the initial number of the first molecule included in the additional sample is at least a threshold difference; and determining a probability that a factor is causing the difference between the initial number of the first molecule included in the sample and the initial number of the first molecule included in the additional sample.

Example 38. A method comprising: obtaining, by at least one computing device of one or more computing devices, sequence data indicating a number of first nucleotide sequences corresponding to a first molecule and a number of second nucleotide sequences corresponding to a second molecule, wherein the sequence data is derived from an amplification product that corresponds to a sample that has undergone an amplification process to increase an initial number of the first molecule and an initial number of the second molecule included in the sample; determining, by at least one computing device of the one or more computing devices, that the first molecule corresponds to a genetic molecule of a biological organism and the second molecule corresponds to a synthetic molecule, the synthetic molecule including first regions that correspond to nucleotide sequences of a biological organism and second regions that include computer-generated nucleotide sequences, and determining, by at least one computing device of the one or more computing devices and based on the initial number of the second molecule included in the sample, an initial number of the genetic molecule included in the sample with a precision of at least 90% and with a lower limit of detection between 1 and 50 of the genetic molecules included in the sample.

Example 39. The method of example 38, comprising: determining, based on the sequencing data and the number of the second nucleotide sequences, the initial number of the genetic molecule included in the sample with a precision of at least 98% and with a lower limit of detection between 1 and 10 of the genetic molecules included in the sample.

Example 40. The method of example 38 or 39, wherein: the nucleotide sequences of the biological organism correspond to primers included in amplification reagents of the amplification process; and the amplification process does not include a real time polymerase chain reaction (PCR) process or a quantitative PCR process.

Example 41. The method of any of examples 38-40, comprising: obtaining the sample from a first source included in an environment, the first source including at least one of a fossil fuel-based petroleum substance or a natural gas substance; obtaining an additional sample from a second source included in the environment, the second source including a rock-based substance; determining a number of the genetic molecule included in the additional sample based on a number of the synthetic molecule included in the additional sample; and determining a difference between the number of the genetic molecule included in the additional sample with respect to the initial number of the genetic molecule included in the sample.

Example 42. The method of any of examples 38-41, comprising: obtaining the sample from a first source included in an environment, the first source including a first portion of a body of an individual; obtaining an additional sample from a second source included in the environment, the second source including a second portion of the body of the individual; determining a number of the genetic molecule included in the additional sample based on a number of the synthetic molecule included in the additional sample; and determining a difference between the number of the genetic molecule included in the additional sample with respect to the initial number of the genetic molecule included in the sample.

Example 43. The method of example 42, comprising: determining that a contaminant is present in the additional sample based on the difference between the number of the genetic molecule included in the additional sample with respect to the initial number of the genetic molecule included in the sample.

Example 44. A method comprising: obtaining a first amount of a first material from a first source included in an environment, the first material including a genetic molecule; obtaining a second amount of a second material from a second source included in the environment, the second material including the genetic molecule; producing a first sample that includes at least a portion of the first amount of the first material, a first amount of one or more amplification reagents, and a first amount of a synthetic molecule, the synthetic molecule including first regions that correspond to primers included in the one or more amplification reagents and second regions that include computer-generated nucleotide sequences; producing a second sample that includes at least a portion of the second amount of the second material, a second amount of the amplification reagents, and a second amount of the synthetic molecule; performing a first amplification process with the one or more amplification reagents to produce a first amplification product for the first sample, the first amplification product including a first amplified number of the genetic molecule that is greater than an initial number of the genetic molecule included in the sample and a first amplified number of the synthetic molecule that is greater than a first initial number of the synthetic molecule included in the first sample; performing a second amplification process with the one or more amplification reagents to produce a second amplification product for the second sample, the second amplification product including a second amplified number of the genetic molecule that is greater than an initial number of the genetic molecule included in the sample and a second amplified number of the synthetic molecule that is greater than a second initial number of the synthetic molecule included in the second sample; performing a first sequencing process to produce first sequence data, the first sequence data indicating nucleotide sequences of molecules included in the first amplification product; performing a second sequencing process to produce second sequence data, the second sequence data indicating nucleotides sequences of molecules included in the second amplification product; determining the first initial number of the genetic molecule included in the first sample based on the first sequence data and the initial number of the synthetic molecule included in the first sample; determining the second initial number of the genetic molecule included in the second sample based on the second sequence data and the initial number of the synthetic molecule included in the second sample; determining a difference between the first initial number of the genetic molecule included in the first sample and the second initial number of the genetic molecule included in the second sample; and performing an analysis based on the difference to determine a probability that a factor of a plurality of factors is causing the difference.

Example 45. The method of example 44, wherein the plurality of factors includes a difference between a first amount of biomass included in the first sample and a second amount of biomass included in the second sample.

Example 46. The method of example 44 or 45, wherein the plurality of factors includes a presence of one or more contaminants in the first sample or the second sample.

Example 47. The method of any of examples 44-46, wherein the plurality of factors includes a difference between one or more first conditions related to the first source and one or more second conditions related to the second source.

Example 48. The method of example 47, wherein the one or more first conditions and the one or more second conditions include at least one of temperature, humidity, or amount of exposure to a range of wavelengths of electromagnetic radiation.

Example 49. The method of any of examples 44-48, wherein the analysis includes a statistical analysis and the analysis is performed based on the difference being at least a threshold difference.

Example 50. The method of any of examples 44-49, wherein the first sequence data includes a first number of nucleotide sequences corresponding to the first amplified number of the genetic molecule included in the first amplification product and a second number of nucleotide sequences corresponding to the first amplified number of the synthetic molecule included in the first amplification product.

Example 51. The method of example 50, comprising determining a ratio corresponding to the first number of nucleotide sequences included in the first sequence data with respect to the initial number of the synthetic molecule included in the first sample; and wherein the first initial number of the genetic molecule included in the first sample is determined based on the ratio.

Example 52. A system comprising: at least one hardware processor; and a computer-readable medium storing instructions that, when executed by the at least one hardware processor, cause the at least one hardware processor to perform operations comprising: obtaining first sequence data indicating a number of first nucleotide sequences corresponding to a genetic molecule and a number of second nucleotide sequences corresponding to a synthetic molecule, wherein the first sequence data is derived from a first amplification product that corresponds to a first sample that has undergone a first amplification process to increase a first initial number of the genetic molecule and a first initial number of the synthetic molecule included in the first sample and wherein the synthetic molecule includes first regions that correspond to primers included in one or more amplification reagents and second regions that include computer-generated nucleotide sequences; obtaining second sequence data indicating a first additional number of first nucleotide sequences corresponding to the genetic molecule and a second additional number of second nucleotide sequences corresponding to the synthetic molecule, wherein the second sequence data is derived from a second amplification product that corresponds to a second sample that has undergone a second amplification process to increase a second initial number of the genetic molecule and a second initial number of the synthetic molecule included in the second sample; determining the first initial number of the genetic molecule included in the first sample based on the first sequence data and the first initial number of the synthetic molecule included in the first sample; determining the second initial number of the genetic molecule included in the second sample based on the second sequence data and the second initial number of the synthetic molecule included in the second sample; determining a difference between the first initial number of the genetic molecule and the second initial number of the genetic molecule; and performing an analysis based on the difference to determine a probability that a factor of a plurality of factors is causing the difference.

Example 53. The system of example 52, wherein the computer-readable medium stores additional instructions that, when executed by the at least one hardware processor, cause the at least one hardware processor to perform additional operations comprising: determining, based on the first sequence data, a first number of nucleotide sequences included in the first sequence data that correspond to the genetic molecule and a second number of nucleotide sequences included in the first sequence data that correspond to the synthetic molecule; and determining the first initial number of the genetic molecule included in the sample is based on the initial number of the synthetic molecule included in the first sample, a volume of the first sample, and the first number of nucleotide sequences included in the sequence data relative to the second number of nucleotide sequences included in the sequence data.

Example 54. The system of example 52 or 53, wherein the computer-readable medium stores additional instructions that, when executed by the at least one hardware processor, cause the at least one hardware processor to perform additional operations comprising: determining a number of third nucleotide sequences included in the first sequence data that correspond to an additional molecule; and determining an initial number of the additional molecule included in the first sample based on the first initial number of the synthetic molecule included in the first sample, the volume of the first sample, and the number of third nucleotide sequences included in the first sequence data relative to the number of second nucleotide sequences of the synthetic molecule included in the first sequence data.

Example 55. The system of example 54, wherein the computer-readable medium stores further instructions that, when executed by the at least one hardware processor, cause the at least one hardware processor to perform further operations comprising: determining a correlation between the initial number of the additional molecule included in the first sample and the first initial number of the synthetic molecule included in the first sample; determining that the correlation satisfies one or more threshold criteria; and determining that the additional molecule is a contaminant.

Example 56. The system of any of examples 51-55, wherein the computer-readable medium stores additional instructions that, when executed by the at least one hardware processor, cause the at least one hardware processor to perform additional operations comprising: performing a comparison of a nucleotide sequence of the genetic molecule to one or more nucleotide sequences included in a library of nucleotide sequences, the library of nucleotide sequences including a plurality of nucleotide sequences that correspond to a plurality of biological organisms; and determining, based on the comparison, a biological organism of the plurality of biological organisms that corresponds to the genetic molecule.

Example 57. The system of example 56, wherein the computer-readable medium stores further instructions that, when executed by the at least one hardware processor, cause at least one hardware processor to perform further operations comprising: determining, based on the first sequence data, a number of additional nucleotide sequences included in the first sequence data that correspond to an additional genetic molecule included in the first sample; performing an additional comparison of an additional nucleotide sequence of the additional genetic molecule to the one or more nucleotide sequences included in the library of nucleotide sequences; determining, based on the additional comparison, a second additional biological organism of the plurality of additional biological organisms that corresponds to the additional genetic molecule; and determining that the biological organism and the additional biological organism are present in environments where at least one of a fossil fuel-based petroleum substance or natural gas are located.

Example 58. The system of any of examples 51-57, wherein the computer-readable medium stores additional instructions that, when executed by the at least one hardware processor, cause the at least one hardware processor to perform additional operations comprising: obtaining additional first samples from the first source over a period of time, the additional first samples including substantially a same amount of the synthetic molecule; obtaining additional second samples from the second source over the period of time, the additional second samples including substantially the same amount of the synthetic molecule; determining amounts of the genetic molecule included in the additional first samples and in the additional second samples based on amounts of the synthetic molecule included in the additional first samples and the additional second samples; determining changes to amounts of the genetic molecule included in at least one of the additional first samples or the additional second samples over the period of time; and performing an analysis to determine a probability that an additional factor of the plurality of factors is causing the changes to the amounts of the genetic molecule included in the at least one of the additional first samples or the additional second samples.

Example 59. A method comprising: obtaining first sequence data indicating a number of first nucleotide sequences corresponding to a genetic molecule and a number of second nucleotide sequences corresponding to a synthetic molecule, wherein the first sequence data is derived from a first amplification product that corresponds to a first sample that has undergone a first amplification process to increase a first initial number of the genetic molecule and a first initial number of the synthetic molecule included in the first sample and wherein the synthetic molecule includes first regions that correspond to nucleotide sequences of a gene of a biological organism and second regions that include machine-generated nucleotide sequences; obtaining second sequence data indicating a first additional number of first nucleotide sequences corresponding to the genetic molecule and a second additional number of second nucleotide sequences corresponding to the synthetic molecule, wherein the second sequence data is derived from a second amplification product that corresponds to a second sample that has undergone a second amplification process to increase a second initial number of the genetic molecule and a second initial number of the synthetic molecule included in the second sample; determining the first initial number of the genetic molecule included in the first sample based on the first sequence data and the first initial number of the synthetic molecule included in the first sample; determining the second initial number of the genetic molecule included in the second sample based on the second sequence data and the second initial number of the synthetic molecule included in the second sample; determining a difference between the first initial number of the genetic molecule and the second initial number of the genetic molecule; and performing an analysis based on the difference to determine a probability that a factor of a plurality of factors is causing the difference.

Example 60. The method of example 59, comprising: determining a first amount of biomass for the first sample, the first amount of biomass corresponding to a first amount of a first substance included in the first sample that includes one or more biological organisms and the genetic molecule corresponding to an additional biological organism included in the one or more biological organisms; and determining a second amount of biomass for the second sample, the second amount of biomass corresponding to a second amount of a second substance included in the second sample that includes one or more additional biological organisms and the additional biological organism being included in the one or more additional biological organisms.

Example 61. The method of example 60, comprising: determining a difference between the first amount of biomass and the second amount of biomass; and determining that the difference between the first initial number of the genetic molecule and the second initial number of the genetic molecule corresponds to the difference between the first amount of biomass and the second amount of biomass.

Example 62. The method of any of examples 59-61, comprising: determining that the difference between the first initial number of the genetic molecule and the second initial number of the genetic molecule corresponds to a contaminant included in the first sample.

Example 63. The method of any of examples 59-62, wherein: the nucleotide sequences included in the first regions of the synthetic molecule correspond to primers included in one or more amplification reagents used in the first amplification process and the second amplification process; and the first amplification process and the second amplification process utilize a polymerase chain reaction (PCR) technique that does not include real-time PCR or quantitative PCR.

1. A method comprising:
obtaining an amount of a material from a source;
extracting a genetic molecule from the amount of the material, the genetic molecule having a first sequence of nucleotides;
generating, by one or more computing devices, first data indicating one or more sequences of nucleotides;
generating, by at least one computing device of the one or more computing devices, second data indicating a second sequence of nucleotides for a synthetic molecule, the synthetic molecule including first regions that include nucleotide sequences of a biological organism and second regions that include additional nucleotide sequences selected from the one or more sequences of nucleotides included in the first data;
producing a volume of a mixture that includes a first portion having an amount of the synthetic molecule and a second portion that includes one or more amplification reagents;
producing a plurality of samples from the mixture, individual samples of the plurality of samples including a portion of the volume of the mixture, and an additional portion that includes an amount of the genetic molecule;
performing an amplification process to produce an amplification product for a sample of the plurality of samples, wherein the amplification product includes an amplified number of the genetic molecule and an amplified number of the synthetic molecule that is greater than an initial number and an initial number of the synthetic molecule included in the sample;
performing a sequencing process to determine nucleotide sequences of molecules included in the amplification product;
obtaining, by at least one computing device of the one or more computing devices and based on the sequencing process, sequence data indicating the nucleotide sequences of the molecules included in the amplification product;
determining, by at least one computing device of the one or more computing devices and based on the sequence data, a first number of nucleotide sequences included in the sequence data that correspond to the genetic molecule and a second number of nucleotide sequences included in the sequence data that correspond to the synthetic molecule; and
determining, by at least one computing device of the one or more computing devices, the initial number of the genetic molecule included in the sample based at least partly on a number of the synthetic molecule included in the sample, a volume of the sample, and the first number of nucleotide sequences included in the sequence data relative to the second number of nucleotide sequences included in the sequence data.

2. The method of claim 1, wherein:
individual samples of the plurality of samples have a volume from 10 microliters to 500 microliters;
the amplification process includes a polymerase chain reaction (PCR) or multiple displacement amplification (MDA) technique;
the nucleotide sequences of the biological organism correspond to primers included in the one or more amplification reagents; and
the nucleotide sequences of the biological organism are selected from conserved regions of the ribosomal ribonucleic acid gene (rRNA gene).

3. The method of claim 1, wherein the genetic molecule includes deoxyribonucleic acid (DNA) or ribonucleic acid (RNA) shared with additional biological organisms and the method comprising:
implementing one or more pseudo-random number generators to generate the first data indicating the one or more sequences of nucleotides; and
dividing a nucleotide sequence generated using the one or more pseudo-random number generators into a plurality of segments to generate a plurality of sequences of nucleotides included in the first data.

4. The method of claim 1, wherein the amount is a first amount, the sample is a first sample, the material is a first material, the source is a first source, and the first source is located in an environment, and the method comprising
obtaining a second amount of a second material from a second source included in the environment, the second material including the genetic molecule
producing a second sample that includes at least a portion of the second amount of the second material, the one or more amplification reagents, and an amount of the synthetic molecule;
performing an additional amplification process with the one or more amplification reagents to produce an additional amplification product for the second sample, the additional amplification product including an additional amplified number of the genetic molecule that is greater than an initial number of the genetic molecule included in the second sample and an additional amplified number of the synthetic molecule that is greater than an additional initial number of the synthetic molecule included in the second sample;
performing an additional sequencing process to produce additional sequence data, the additional sequence data indicating nucleotides sequences of molecules included in the additional amplification product;
determining the initial number of the genetic molecule included in the second sample based on the second sequence data and the initial number of the synthetic molecule included in the second sample;
determining a difference between the initial number of the genetic molecule included in the first sample and the initial number of the genetic molecule included in the second sample; and
performing an analysis based on the difference to determine a probability that a factor of a plurality of factors is causing the difference.

5. The method of claim 1, wherein the volume of the mixture includes a third portion that includes an amount of an additional molecule, and the method comprises:
determining, by at least one computing device of the one or more computing devices and based on the sequence data, a third number of nucleotide sequences included in the sequence data that correspond to the additional molecule; and
determining, by at least one computing device of the one or more computing devices, an initial number of the additional molecule included in the sample based at least partly on the number of the synthetic molecule included in the sample, the volume of the sample, and the third number of nucleotide sequences included in the sequence data relative to the second number of nucleotide sequences included in the sequence data.

6. The method of claim 5, comprising:
determining, by at least one computing device of the one or more computing devices, a correlation between the number of the synthetic molecule included in the sample and the initial number of an additional molecule included in the sample;
determining, by at least one computing device of the one or more computing devices, that the correlation satisfies one or more threshold criteria;
determining, by at least one computing device of the one or more computing devices, that the additional molecule is a contaminant included in the one or more amplification reagents.

7. The method of claim 5, comprising:
determining, by at least one computing device of the one or more computing devices, that the third number of the additional molecule is greater than a threshold number; and
determining, by at least one computing device of the one or more computing devices, that the additional molecule is another genetic molecule included in the sample.

8. The method of claim 1, comprising determining the number of the synthetic molecule included in the sample based on a number of samples derived from the volume of the mixture and the amount of the synthetic molecule included in the volume of the mixture.

9. (canceled)

10. A system comprising:
at least one hardware processor; and
a computer-readable medium storing instructions that, when executed by the at least one hardware processor, cause the at least one hardware processor to perform operations comprising:
obtaining sequence data indicating nucleotide sequences of molecules included in an amplification product, the amplification product corresponding to a sample that has undergone an amplification process to increase an initial number of molecules included in the sample;
determining, based on the sequence data, a first number of nucleotide sequences included in the sequence data that correspond to a first genetic molecule included in the sample and a second number of nucleotide sequences included in the sequence data that correspond to a synthetic molecule included in the sample, the synthetic molecule including first regions that include first nucleotide sequences of a biological organism and second regions that include second nucleotide sequences selected from one or more machine-generated sequences of nucleotides; and
determining, an initial number of the genetic molecule included in the sample based at least partly on an initial number of the synthetic molecule included in the sample, a volume of the sample, and the first number of nucleotide sequences included in the sequence data relative to the second number of nucleotide sequences included in the sequence data.

11. The system of claim 10, wherein the computer-readable medium stores additional instructions that, when executed by the at least one hardware processor, cause the at least one hardware processor to perform additional operations comprising:
performing a comparison of a nucleotide sequence of the genetic molecule to one or more nucleotide sequences included in a library of nucleotide sequences, the library of nucleotide sequences including a plurality of nucleotide sequences that correspond to a plurality of additional biological organisms; and
determining, based on the comparison, an additional biological organism of the plurality of additional biological organisms that corresponds to the genetic molecule.

12. The system of claim 11, wherein the computer-readable medium stores additional instructions that, when executed by the at least one hardware processor, cause the at least one hardware processor to perform additional operations comprising:
determining, based on the sequence data, a third number of nucleotide sequences included in the sequence data that correspond to an additional genetic molecule included in the sample;
performing a comparison of an additional nucleotide sequence of the additional genetic molecule to the one or more nucleotide sequences included in the library of nucleotide sequences;
determining, based on the comparison, a second additional biological organism of the plurality of additional biological organisms that corresponds to the additional genetic molecule; and
determining that the additional biological organism and the second additional biological organism are present in environments where at least one of crude oil, natural gas, and formation water are located.

13. The system of claim 10, wherein the computer-readable medium stores further instructions that, when executed by the at least one hardware processor, cause the at least one hardware processor to perform further operations comprising:
determining, based on the sequence data, a third number of nucleotide sequences included in the sequence data that correspond to an additional molecule;
determining a correlation between the third number of nucleotide sequences and the second number of nucleotide sequences;
determining that the correlation satisfies one or more threshold criteria; and
determining that the additional molecule is a contaminant included in the one or more amplification reagents.

14. The system of claim 10, wherein the initial number of the genetic molecule is determined with a precision of at least 95% and with a lower limit of detection between 1 and of the genetic molecule included in the sample.

15. A method comprising:
obtaining, by one or more computing devices, sequence data indicating nucleotide sequences of molecules included in an amplification product, the amplification product corresponding to a sample that has undergone an amplification process to increase an initial number of molecules included in the sample;
determining, by at least one computing device of the one or more computing devices and based on the sequence data, a first number of nucleotide sequences included in the sequence data that correspond to a genetic molecule included in the amplification product and a second number of nucleotide sequences included in the sequence data that correspond to a synthetic molecule included in the amplification product, the synthetic molecule including first regions that include nucleotide sequences of a biological organism and second regions that include nucleotide sequences selected from one or more machine-generated sequences of nucleotides;
generating a function to determine a number of the genetic molecule included in the sample; and
determining, by at least one computing device of the one or more computing devices, the number of the genetic molecule included in the sample based on the number of the synthetic molecule included in the sample and the first number of nucleotide sequences included in the sequence data.

16. The method of claim 15, comprising:
determining, based on a presence of the genetic molecule in the sample, that one or more biochemical reactions have taken place in an environment from which the sample was obtained, wherein the one or more biochemical reactions include at least one of a nitrate reducing reaction, a sulfate reducing reaction, methanogenesis, a hydrocarbon conversion reaction, or a biosurfactant generating reaction.

17. The method of claim 15, comprising:
extracting the genetic molecule from a cell included in an amount of a fluid obtained from a subterranean environment that stores at least one of a fossil fuel-based petroleum substance or natural gas.

18. The method of claim 15, comprising:
performing comparisons between a nucleotide sequence of the genetic molecule with respect to a plurality of additional nucleotide sequences that are associated with a plurality of individuals;
determining, based on the comparisons, a threshold amount of identity between the nucleotide sequence of the genetic molecule and an additional nucleotide sequence of the plurality of additional nucleotide sequences; and
identifying an individual of the plurality of individuals that corresponds to the additional nucleotide sequence.

19. The method of claim 15, comprising:
obtaining an amount of a material from an environment;
extracting a cell from the material that includes the genetic molecule;
performing comparisons between a nucleotide sequence of the genetic molecule with respect to a plurality of additional nucleotide sequences that are associated with contaminants;
determining, based on the comparisons, a threshold amount of identity between the nucleotide sequence of the genetic molecule and an additional nucleotide sequence of the plurality of additional nucleotide sequences; and
identifying a contaminant of the environment, wherein the contaminant corresponds to the additional nucleotide sequence.

20. The method of claim 15, comprising:
identifying a first barcode sequence included in a first nucleotide sequence;
determining, based on the first barcode sequence, that the first nucleotide sequence corresponds to a first molecule;
identifying a second barcode sequence included in a second nucleotide sequence;
determining, based on the second barcode sequence, that the second nucleotide sequence corresponds to a second molecule; producing a first group of nucleotide sequences that include the first barcode sequence;
producing a second group of nucleotide sequences that include the second barcode sequence;
determining the number of the first nucleotide sequences based on a number of nucleotide sequences included in the first group; and
determining the number of the second nucleotide sequences based on a number of nucleotide sequences included in the second group.

21.-25. (canceled)

26. The method of claim 4, wherein the plurality of factors includes:
a difference between a first amount of biomass included in the first sample and a second amount of biomass included in the second sample; or
a difference between one or more first conditions related to the first source and one or more second conditions related to the second source, the one or more first conditions and the one or more second conditions include at least one of temperature, humidity, or amount of exposure to a range of wavelengths of electromagnetic radiation.
